(19) United States
(12) Patent Application Publication
(10) Pub. No.: US 2026/0038304 A1
(43) Pub. Date: Feb. 5, 2026

(54) METHOD AND SYSTEM FOR DETERMINING EFFICACY OF TREATMENT BY A PREDETERMINED SUBSTANCE

(71) Applicant: YEDA RESEARCH AND DEVELOPMENT CO. LTD., Rehovot (IL)

(72) Inventors: Takashi KAWASHIMA, Tel Aviv (IL); Rani BARBARA, Rehovot (IL); Dotan BRAUN, Rehovot (IL); Ayelet ROSENBERG, Rehovot (IL)

(21) Appl. No.: 19/351,050

(22) Filed: Oct. 6, 2025

Related U.S. Application Data
(63) Continuation of application No. PCT/IL2024/050363, filed on Apr. 11, 2024.
(60) Provisional application No. 63/614,639, filed on Dec. 25, 2023.

(30) Foreign Application Priority Data
Apr. 13, 2023 (IL) 302100

(51) Int. Cl. G06V 40/20; G06T 7/246; G06V 10/62; G06V 10/764; G06V 10/77; G06V 40/10; G16H 20/10

(52) U.S. Cl. CPC G06V 40/20; G06T 7/246; G06V 10/62; G06V 10/764; G06V 10/7715; G06V 40/10; G16H 20/10; G06T 2207/20081

(57) ABSTRACT

A system and method of determining efficacy of treatment by at least one processor may include receiving, from at least one camera, images depicting motion of an animal that may be treated with a predetermined substance of interest. Said processor may extract from the images, a plurality of motion features representing motion of at least one specific body part of the animal, and apply a dimensionality reduction algorithm on the plurality of motion features, to obtain a latent vector representing the plurality of motion features in a latent space. The latent vector may include a plurality of latent features. Said processor may subsequently calculate a value of a behavioral indicator, representing a behavior of the animal, based on the latent features of the latent vector, and determine efficacy of the treatment based on the behavioral indicator value.

### CROSS REFERENCE TO RELATED APPLICATIONS

This application is a Bypass Continuation of PCT International Application No. PCT/IL2024/050363, having international filing date of Apr. 11, 2024, which claims the benefit of priority of Israeli Patent Application No. 302100, filed Apr. 13, 2023, titled “Method and system for determining efficacy of treatment by a predetermined substance”; and U.S. Provisional Patent Application No. 63/614,639, filed Dec. 25, 2023, titled “Method and system for determining efficacy of treatment by a predetermined substance”. The contents of the abovementioned applications are all incorporated herein by reference in their entirety.

### FIELD OF THE INVENTION

The present invention relates generally to the field of image analysis. More specifically, the present invention relates to the assessment of treatment efficacy by a substance of interest, as determined through the analysis of treatment-related imagery.

### BACKGROUND OF THE INVENTION

Serotonergic psychedelics are emerging therapeutics for psychiatric disorders, yet their underlying mechanisms of action in the brain remain largely elusive. Zebrafish have evolutionarily conserved serotonergic circuits and subcortical targets such as the brainstem regions and the cerebellum, providing a promising model for studying the subcortical effects of serotonergic drugs.

Mood-related mental disorders cast significant socioeconomic impacts on modern societies, and 5% of adults are estimated to suffer from depression globally1. The serotonin theory of depression emerged soon after the discovery of the serotonergic system in the brain in the 1960s. Since then, the serotonergic system has been a therapeutic target for major depression, obsessive-compulsive disorders, and other psychiatric disorders. Current medication regimens based on serotonin-selective reuptake inhibitors (SSRIs) have limited efficacies in terms of quickness of therapeutic effects and final remission rates, calling for a better understanding of neural mechanisms for mood-related behavioral alternation and its pharmaceutical rescue.

Recent resurgence of the use of hallucinogenic drugs as fast-acting antidepressants has opened new opportunities for the research of neural circuit mechanisms critical for the treatment of mood-related disorders. Psilocybin, a psychedelic compound, originates in the genus of gilled mushrooms Psilocybe and acts as a potent agonist for a family of serotonin receptors. Psilocybin is effective for clinical cases of treatment-resistant depression, and only a few doses can have lasting effects on depression symptoms for months or even up to a year. These reported therapeutic effects are markedly different from the short-lasting effects of other classes of psychedelics such as ketamine, making psilocybin and its derivatives a promising class of drugs for treating mood-related disorders.

There is currently limited understanding of the mechanisms underlying the therapeutic effects of psilocybin. Human brain imaging studies show that psilocybin alters the functional connectivity within the default mode network, including the prefrontal cortex and posterior cingulate cortex. Microscopic observations of the prefrontal cortex in mice suggest that such changes might occur from the induction of new excitatory synapses. There have also been efforts to derive HTR2 agonists that can prevent stress-induced behavioral changes without causing hallucination-like behaviors. Very few studies have focused on changes in subcortical structures such as the brainstem and the cerebellum, which are enriched for serotonin receptors. Roles of the cerebellum have been implicated in mood-related disorders, and psilocybin affects cerebellar neural activity in humans. In general, neural dynamics in these subcortical structures have been challenging to investigate in mammals.

### SUMMARY OF THE INVENTION

Larval zebrafish may serve as a model animal for studying subcortical structures that are evolutionarily conserved across vertebrates. The zebrafish's small size, optical transparency, and genetic accessibility allow optical recording of neural activity across the whole brain at a single-cell resolution. It has a conserved raphe serotonergic system in the hind/midbrain in addition to teleost-specific serotonergic nuclei in the hypothalamus, that allow detailed investigation of the working principles of serotonergic neurons during behavior.

Larval zebrafish have also been used to screen the behavioral effect of stress exposure, antidepressants, and genetic mutations. However, few published studies have examined the behavioral effects of psychedelics in larval zebrafish, and, to date, there have been no published data on the effects of psilocybin. Such scarcity of behavioral insights impedes further research into their actions in neural circuit dynamics.

Accordingly, there is a need for a method and system for determining efficacy of treatment, as well as a method of screening for a compound suitable for treating a psychological state in a subject in need thereof, which would contribute to the improvement of the abovementioned technological field by providing highly reliable tool for evaluation of the behavioral effect in a subject (e.g., larval zebrafish) treated with a substance of interest, based on treatment-related imagery.

The inventors have developed a wide-field behavioral tracking system for larval zebrafish and investigated the effects of substances of interest such as psilocybin, which is a psychedelic serotonin receptor agonist. Machine learning analyses of precise body kinematics identified latent behavioral states reflecting spontaneous exploration, visually driven rapid swimming, and irregular swim patterns following stress exposure. Using this method, the inventors have identified two main behavioral effects of acute psilocybin treatment: [i] increased rapid swimming in the absence of visual stimuli and [ii] prevention of irregular swim patterns following stress exposure. Together, these effects indicate that psilocybin induces a brain state that is both stimulatory and anxiolytic. These findings pave the way for using larval zebrafish to elucidate subcortical mechanisms underlying the behavioral effects of serotonergic psychedelics.

Aspects of the invention may include a method of determining efficacy of treatment by at least one processor. The method of determining efficacy of treatment may include: receiving, from at least one camera, images depicting motion of an animal, wherein said animal is treated with a predetermined substance; extracting, from said images, a plurality of motion features, each representing a specific quantified motion characteristic of at least one specific body part of the animal; applying a dimensionality reduction algorithm on the plurality of motion features, to obtain a latent vector representing the plurality of motion features in a latent space, wherein the latent vector comprises a plurality of latent features; calculating a value of a behavioral indicator, representing a behavior of the animal, based on the latent features of the latent vector; and determining efficacy of the treatment based on the behavioral indicator value.

According to some embodiments, the animal may be a fish, and the body parts may include, for example a head of the fish, a tail of the fish, an eye of the fish and a heart of the fish.

According to some embodiments, the plurality of motion features may include, for example tail motion features such as a frequency of tail motions, an amplitude of tail motions, an angle of tail motions, a number of tail motions in a predetermined timeframe, a balance of tail motions between a left side and a right side of the fish.

Additionally, or alternatively, the plurality of motion features may include, for example head motion features such as a frequency of head motions, an angle of head motions, and a number of head motions within a predefined timeframe.

Additionally, or alternatively, the plurality of motion features may include, for example a frequency of fin motions, an amplitude of fin motions, an angle of eye motions, and a frequency of eye motions.

Additionally, or alternatively, the plurality of motion features may include, for example swim interval features such as a duration of a swim episode, and an interval between swim episodes.

In some embodiments, the method of determining efficacy of treatment may further include applying a pretrained, first machine learning (ML) based model, to classify the latent vector to one or more classes of movement patterns; and calculating the behavioral indicator value based on said classification.

In some embodiments, the method of determining efficacy of treatment may further include training the first ML based model. The training may include: receiving a training set, comprising a plurality of time-based sequences of one or more latent vectors; obtaining a plurality of movement pattern annotations, corresponding to the plurality of latent vector sequences; and using the plurality of movement pattern annotations as supervisory data, to train the first ML based model to classify latent vectors of at least one sequence to one or more classes of movement patterns.

According to some embodiments, the classes of movement patterns may include, for example traversal patterns such as short scooting, rapid long scooting, performance of routine turns, performance of C-turns, a pattern of immobility, and patterns of intermittent mobility.

Additionally, or alternatively, the classes of movement patterns may include, for example organ movement patterns such as an eye movement pattern, a heart movement pattern, a fin movement pattern, and a limb movement pattern.

According to some embodiments, the behavioral indicator may include, for example a level of anxiety of the animal, a level of arousal of the animal, a level of responsiveness of the animal to a visual stimulus, a level of responsiveness of the animal to an odor stimulus, a level of responsiveness of the animal to an acoustic stimulus, a level of motoric disability of the animal, a level of appetite of the animal, a level of sleepiness of the animal, and the like.

In some embodiments, determining efficacy of the treatment may include: comparing a pre-treatment value of the behavioral indicator, with a post-treatment value of the behavioral indicator; and calculating a statistical significance value, representing correlation between application of the predetermined substance and the behavioral indicator, based on said comparison, to determine efficacy of the treatment.

In some embodiments, extracting the motion features may include: identifying one or more body parts of the depicted fish in said images; applying a second ML based model on the identified body parts, to do determine locations of specific points of the fish, at sub-pixel resolution; fitting the determined locations in a quadratic curve; quantifying motion of the at least one body part based on said fitting; and calculating a value of at least one motion feature based on said quantification.

In some embodiments, quantifying motion of the at least one body part may be selected from: (i) computing a rate of tail strokes, based on location of one or more specific points of the fish on the quadratic curve; and (ii) computing an amplitude of tail strokes, based on location of one or more specific points of the fish on the quadratic curve.

In some embodiments, the method of determining efficacy of treatment may further include: identifying a sub-pixel centroid location of a head of the depicted fish, and computing the rate of tail strokes, further based on the identified centroid location.

Aspects of the invention may further include a system for determining efficacy of treatment. Embodiments of the system may include at least one camera, configured to obtain images depicting motion of an animal, wherein said animal may be treated with a predetermined substance; a non-transitory memory device wherein modules of instruction code may be stored; and at least one processor associated with the memory device, and configured to execute the modules of instruction code.

Upon execution of said modules of instruction code, the at least one processor may be configured to: receive, from the at least one camera, images depicting motion of the animal, said animal being treated with a predetermined substance of interest; extract, from said images, a plurality of motion features, each representing a specific quantified motion characteristic of at least one specific body part of the animal; and apply a dimensionality reduction algorithm on the plurality of motion features, to obtain a latent vector representing the plurality of motion features in a latent space. The latent vector may include a plurality of latent features.

According to some embodiments, the at least one processor may be configured to apply a pretrained, first machine learning (ML) based model, to classify the latent vector to one or more classes of movement patterns, and calculate the behavioral indicator value based on said classification.

According to some embodiments, the at least one processor may be configured to train the first ML based model by receiving a training set that may include a plurality of time-based sequences of one or more latent vectors; obtaining a plurality of movement pattern annotations, corresponding to the plurality of latent vector sequences; and using the plurality of movement pattern annotations as supervisory data, to train the first ML based model to classify latent vectors of at least one sequence to one or more classes of movement patterns.

The at least one processor may be further configured to calculate a value of a behavioral indicator, representing a behavior of the animal, based on the latent features of the latent vector; and determine efficacy of the treatment based on the behavioral indicator value.

According to some embodiments, the at least one processor may be further configured to determine efficacy of the treatment by comparing a pre-treatment value of the behavioral indicator, with a post-treatment value of the behavioral indicator; and calculating a statistical significance value, representing correlation between application of the predetermined substance and the behavioral indicator, based on said comparison, to determine efficacy of the treatment.

According to some embodiments, the at least one processor may be further configured to extract the motion features by: identifying one or more body parts of the depicted fish in said images; applying a second ML based model on the identified body parts, to do determine locations of specific points of the fish, at sub-pixel resolution; fitting the determined locations in a quadratic curve; quantifying motion of the at least one body part based on said fitting; and calculating a value of at least one motion feature based on said quantification.

According to some embodiments, the at least one processor may be further configured to quantify motion of the at least one body part by computing a rate of tail strokes, based on location of one or more specific points of the fish on the quadratic curve.

Additionally, or alternatively, the at least one processor may be further configured to quantify motion of the at least one body part by computing an amplitude of tail strokes, based on location of one or more specific points of the fish on the quadratic curve.

Additionally, or alternatively, the at least one processor may be further configured to identify a sub-pixel centroid location of a head of the depicted fish, and compute the rate of tail strokes, further based on the identified centroid location.

Aspects of the invention may further include a method of screening for a compound suitable for treating a psychological state in a subject in need thereof by at least one processor. Embodiments of the method may include administering an animal with an effective amount of the compound; measuring a plurality of motion features representing motion of at least one specific body part of the animal; determining a latent vector representing the plurality of motion features in a latent space, wherein the latent vector may include a plurality of latent features; and calculating a value of a behavioral indicator, representing a behavior of the administered animal, based on the latent features of the latent vector. In such embodiments, a behavioral indicator of the animal administered with the compound being equal to, or greater than a pre-determined threshold, may be indicative of the compound being suitable for treating the psychological state in a subject in need thereof.

Additionally, or alternatively, a behavioral indicator of the animal administered with the compound being lower than a pre-determined threshold may be indicative of the compound being unsuitable for treatment.

According to some embodiments, the animal may be a fish, and the administering may be performed via feeding, or via introduction of the compound into a body of water in which the animal resides.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A is a schematic illustration, depicting an experimental setup and analysis pipeline set by the inventors, in which an animal (e.g., a larvae fish) 25, swam in an arena 20AR where visual stimuli were projected underneath. The setup included an imaging device 20, able to acquire high-resolution images 25′ at a high speed (e.g., 290 Hz) across an arena 20AR of up to 90 mm in diameter. According to some embodiments of the invention, data analysis pipelines processed the images to identify the fish loci and body postures by using a deep neural network.

FIG. 1B depicts head centroid trajectories in a small, walled environment (30 mm) and a large, unwalled environment (90 mm), as used by some embodiments of the invention. In the illustrated head centroid trajectories, different swim episodes can be visually separated.

FIG. 1C depicts examples of calculated distributions of head centroids during experiments across tested fish. N=22 and N=20 fish for the small and large dishes, respectively, according to some embodiments of the invention;

FIG. 1D depicts swimming distance per minute during spontaneous exploration and visually induced swimming. Small circles represent individual fish. **, p=0.0081; ***, p=1.7*10⁻⁶ from 2-sample t-test.

FIG. 1E depicts Expanded head centroid trajectories from the outlined central parts of the small arena (left) and the large arena (right) from (B). As can be seen, the large environment facilitates straight swim patterns;

FIG. 1F depicts an example of increase in straight swimming during visually induced swimming in the large arena (right bar of each two paired bars) compared to the small arena (left bar of each two paired bars), ***, p=1.8*10⁻⁶ from 2-sample t-test, where error bars represent standard deviations across tested fish;

FIG. 1G, depicts an example of zebrafish swim patterns in a large environment (90 mm) with a flat floor and a boundary wall, where the left panel shows head centroid trajectories of a single fish, and the right panel shows, expanded head centroid trajectories from the outlined central parts of the large arena on the left;

FIG. 2A depicts swim velocities, tail motion, and head centroid motions during multiple swim episodes, according to some embodiments of the invention;

FIG. 2B depicts motion features (e.g., features of tail motion) and features of traversal (e.g., swimming features, or swim parameters), that may be obtained by embodiments of the invention, for reduced dimensionality analysis such as Independent Component Analysis (ICA).

FIG. 2C depicts an example of latent feature (e.g., ICA: IC1—top bar of each two paired bars; and IC2—bottom bar of each two paired bars) weights, according to some embodiments of the invention;

FIG. 2D represents scatter density plots of various movement patterns or traversal patterns (swim patterns) in a latent (e.g., ICA) space, revealing enriched repertoires of swimming in a large environment, according to some embodiments of the invention;

FIG. 2E depicts annotations of point across an animals body, according to some embodiments of the invention;

FIG. 2F depicts quantification of a tail angle θ, by fitting a quadratic function to annotated points along the tail, according to some embodiments of the invention;

FIG. 2G presents an example of as overlay of tail motions and head centroid motions during a representative swim episode;

FIG. 2H represents validation of accuracy of tail angle quantification by predicting swimming distance based on tail motion parameters, by some embodiments of the invention.

FIG. 2I presents an example of statistical analyses of latent feature 140LF ICA1 and latent feature 140LF ICA2, according to some embodiments of the invention;

FIG. 2J presents an example of statistical analyses of individual traversal motion features (also referred to as swim parameters), according to some embodiments of the invention;

FIGS. 3A-3H represent aspects of effect of a predetermined treatment (e.g., a drug of interest), on motion features and movement patterns of the examined animal, according to some embodiments of the invention;

FIG. 3I depicts unbiased homology analyses of protein sequences of all serotonin receptors revealed conserved major types and subtypes between zebrafish and humans;

FIG. 3J depicts effect of psilocybin treatment on swimming distances during spontaneous exploration and visually driven swimming, as observed by the inventors;

FIG. 3K presents swim trajectories of control, fluoxetine-treated, and fluvoxamine-treated fish during visually driven behaviors, according to some embodiments.

FIG. 3L presents swimming distance per minute (left), tail frequency (center) and average tail angle (right) of control (N=21), fluoxetine-treated (N=17 for 1 μM, N=20 for 10 μM) and fluvoxamine-treated fish (N=20 for 2.5 μM, N=20 for 12.9 μM, N=12 for 25 μM), according to some embodiments.

FIG. 4A depicts behavioral paradigms for psilocybin treatment and acute cold stress exposure;

FIG. 4B depicts swimming distances during spontaneous exploration and visually driven swimming. N=14 (C), 14 (S), 16 (P) and 15 (P/S) fish. *, p=0.021 from Tukey's post-hoc test between the control and P/S conditions after one-way ANOVA detected significant differences among groups for spontaneous swimming distance.

FIG. 4C depicts acute cold stress exposure induced zigzag swim patterns during visually driven swimming, and psilocybin prevented such stress-induced behavioral changes. Shaded boxes indicate changed temperature or drug conditions.

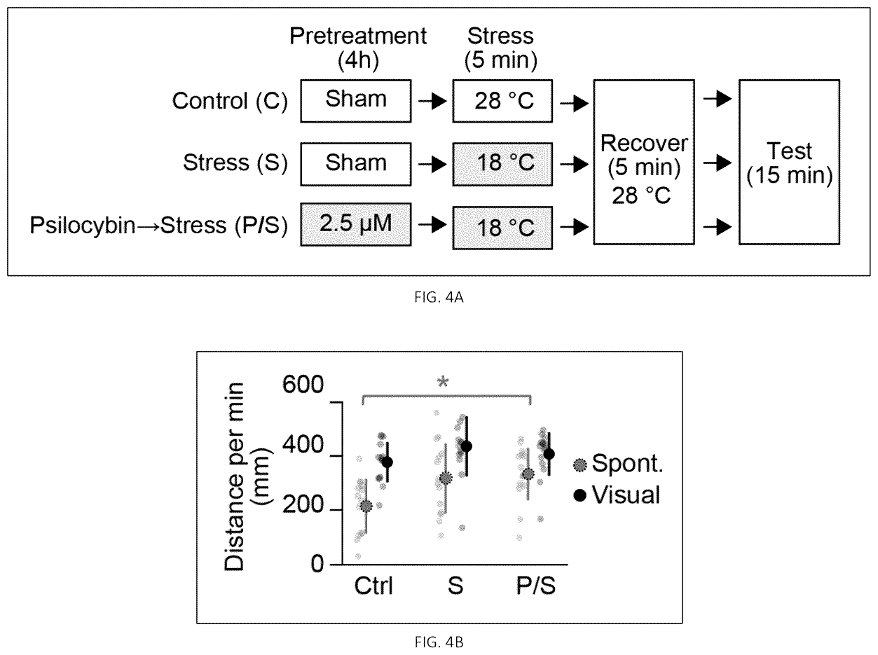

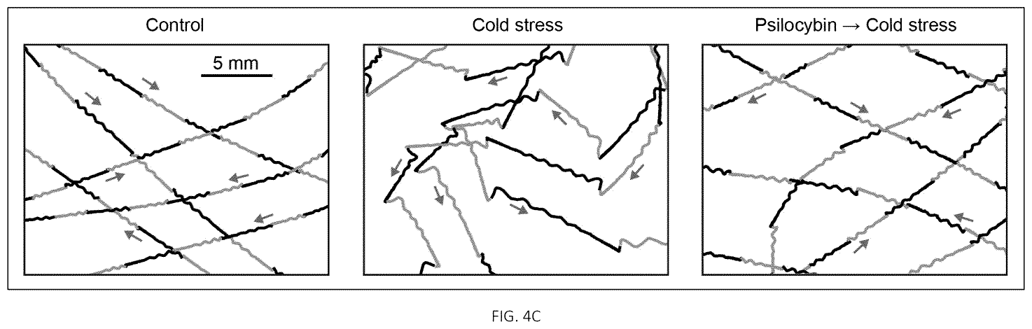

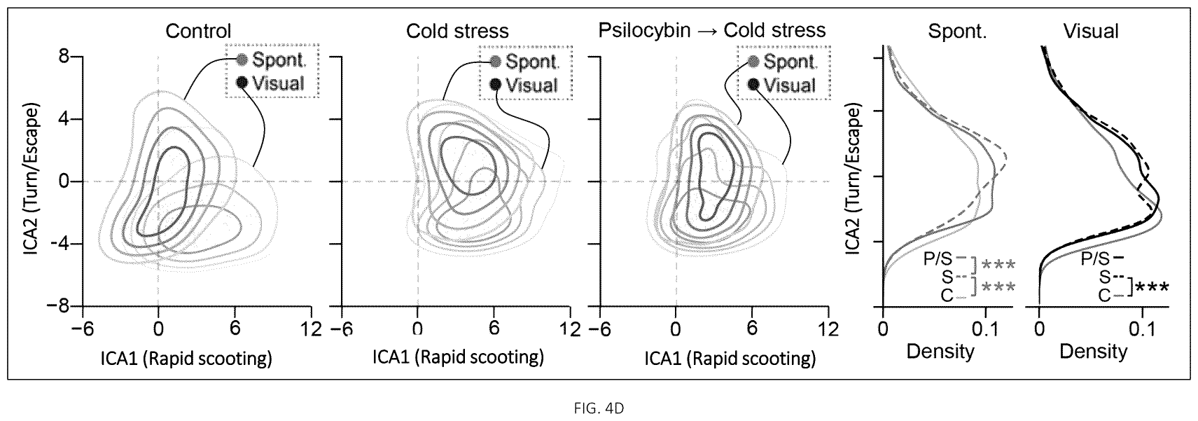

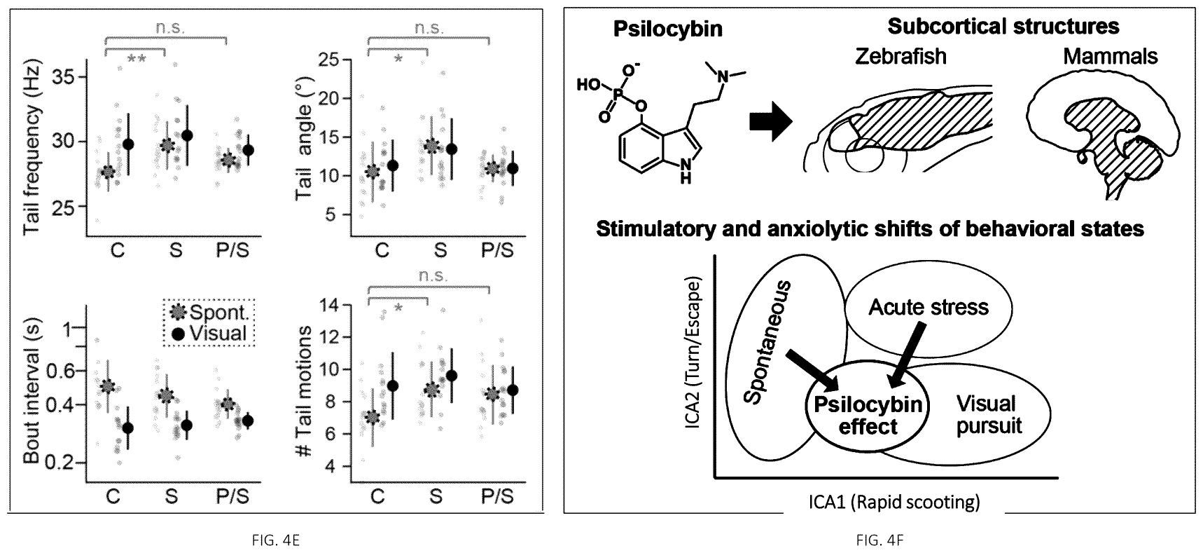

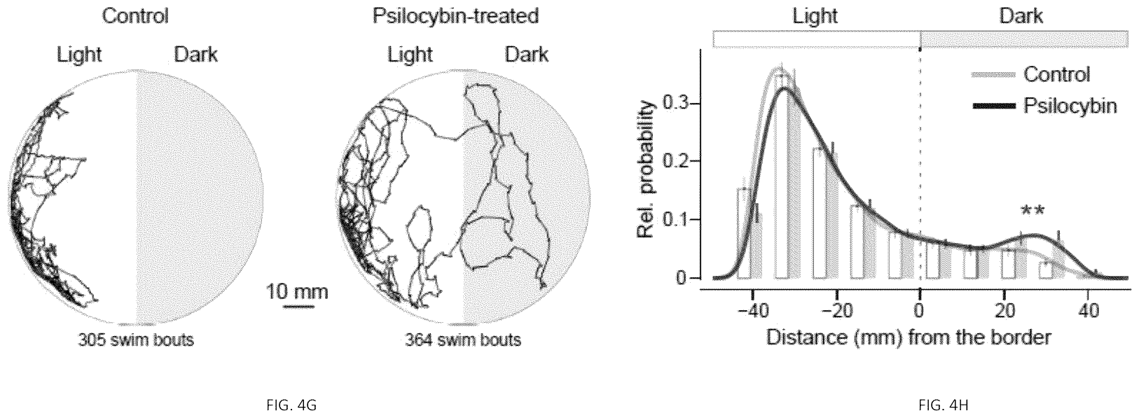

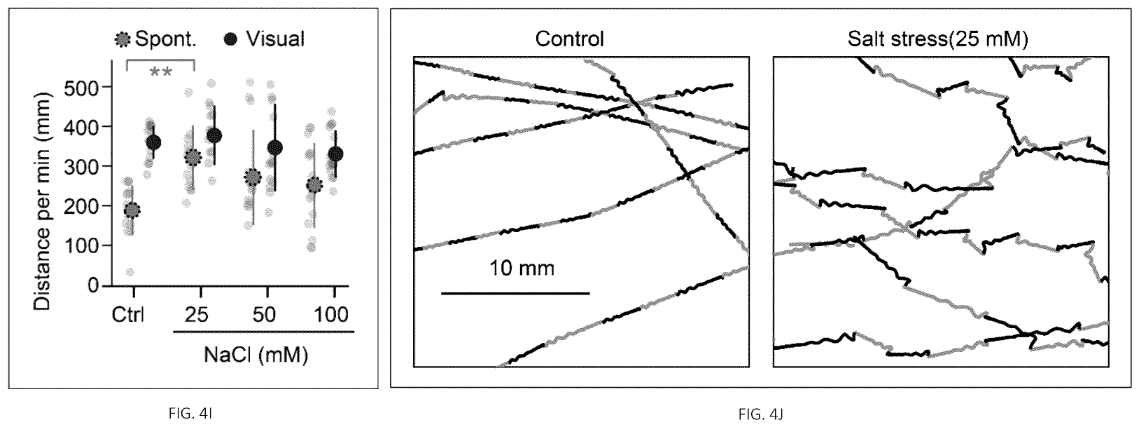

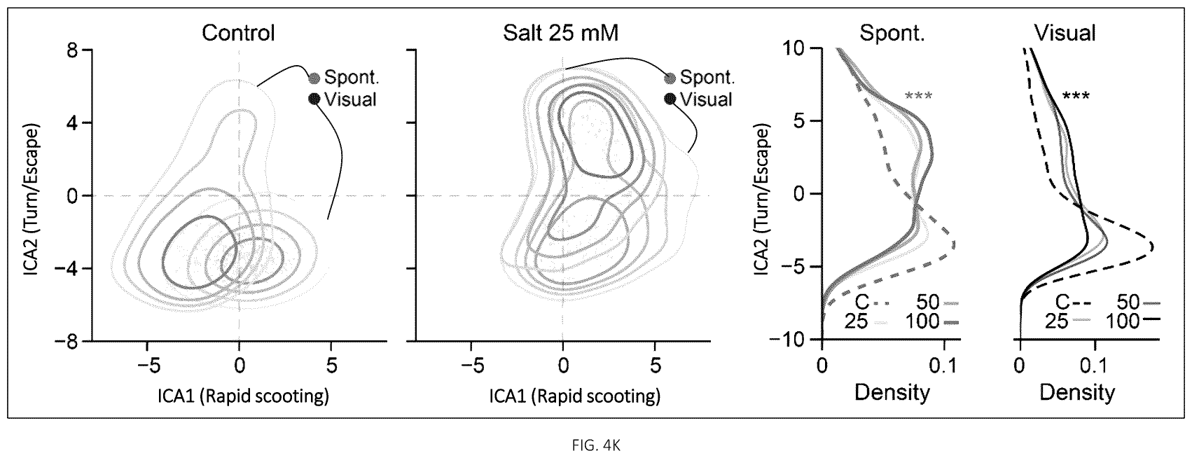

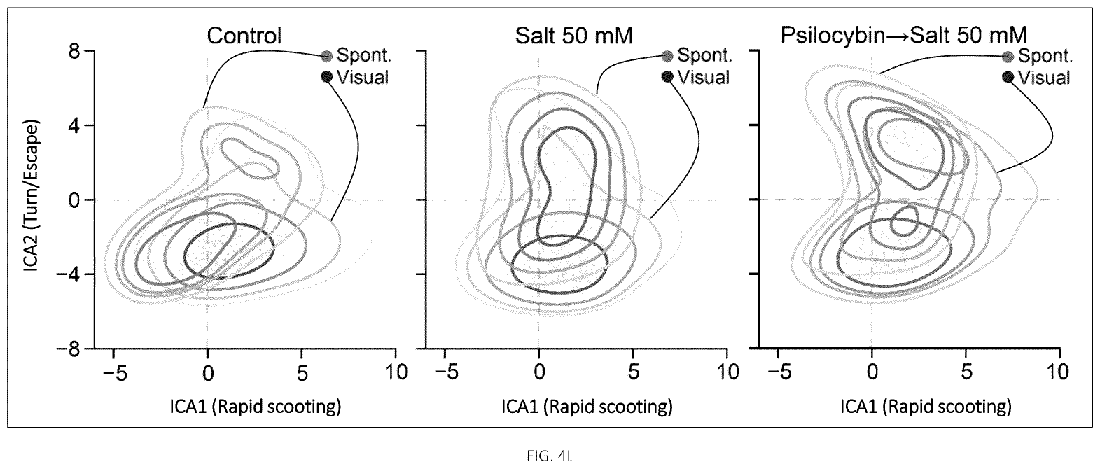

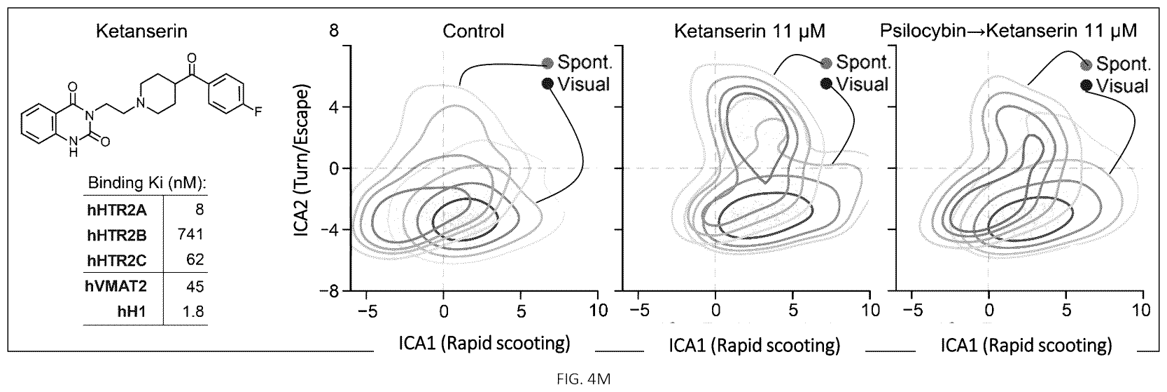

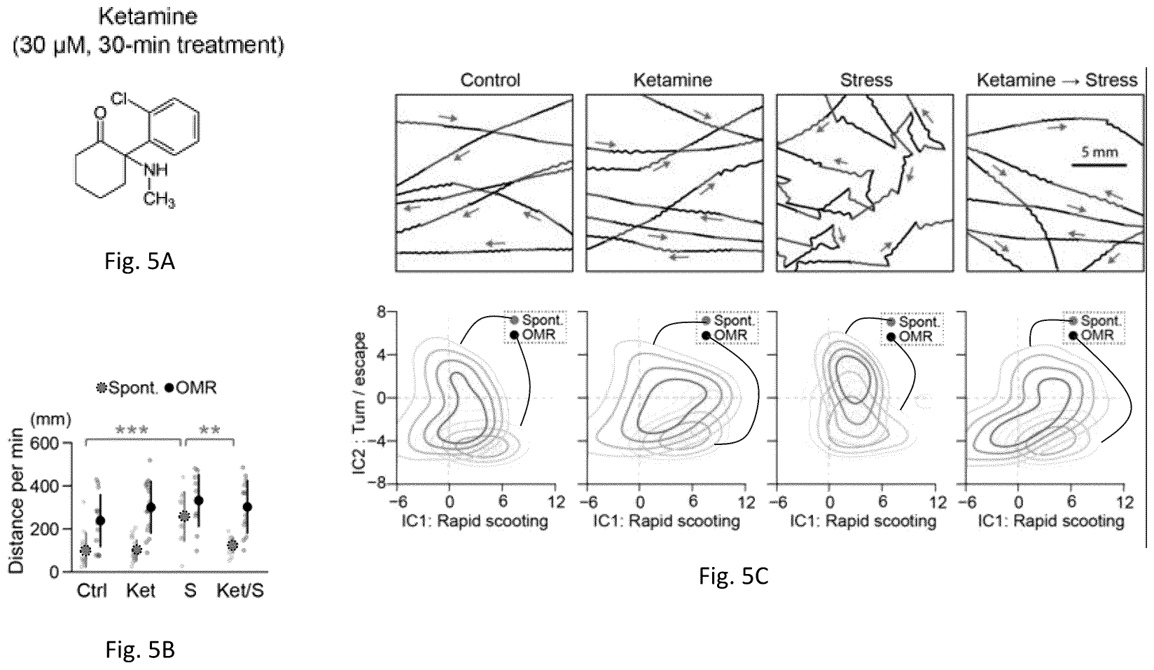

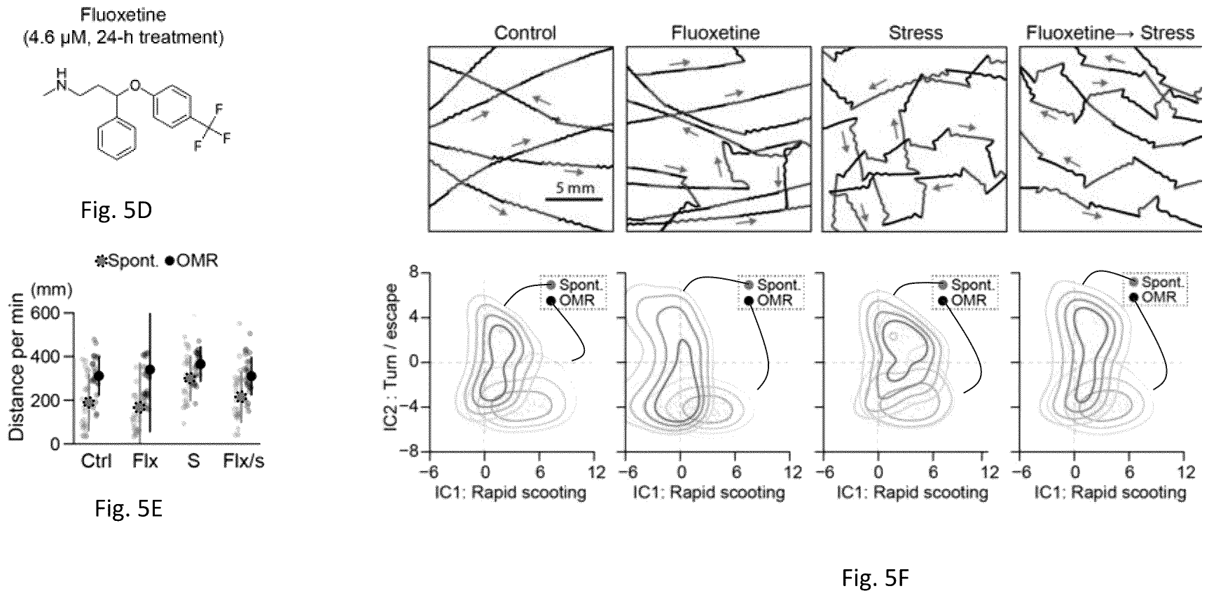

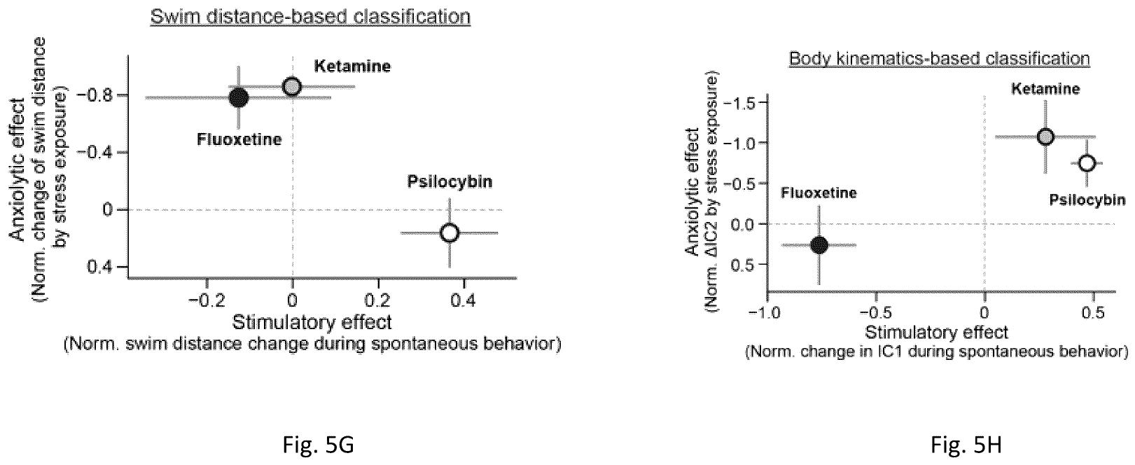

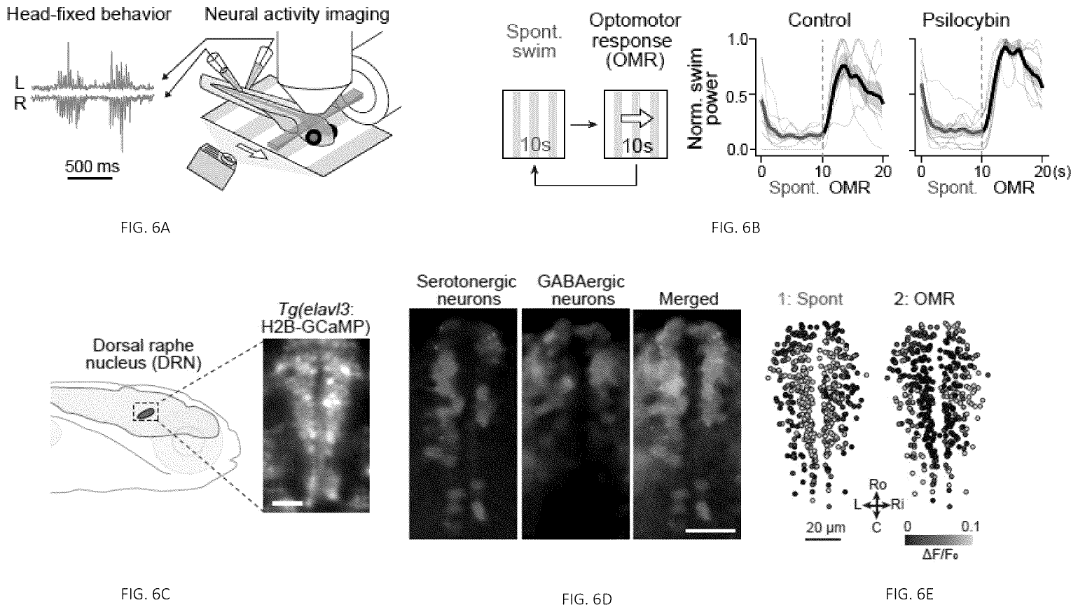

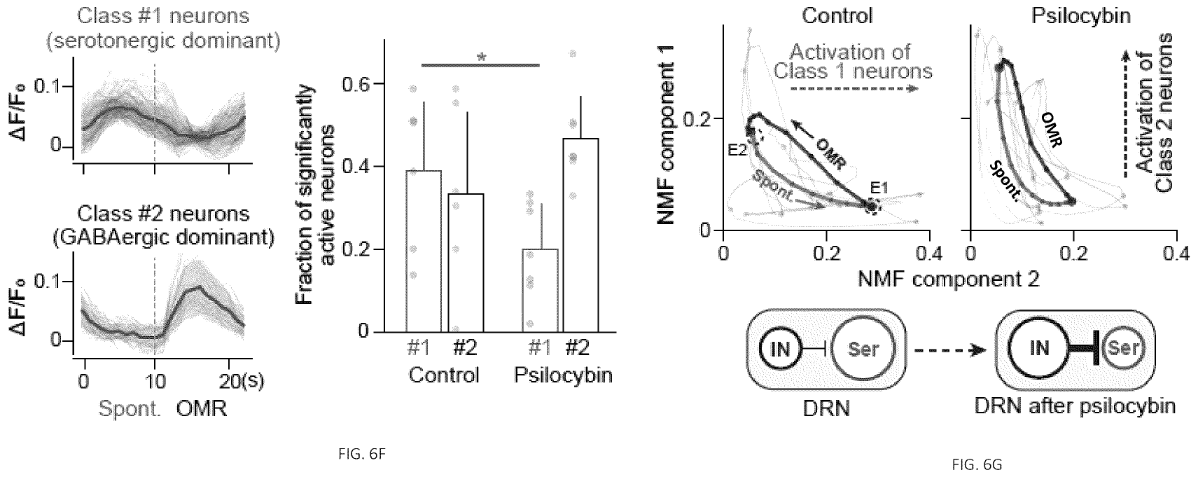

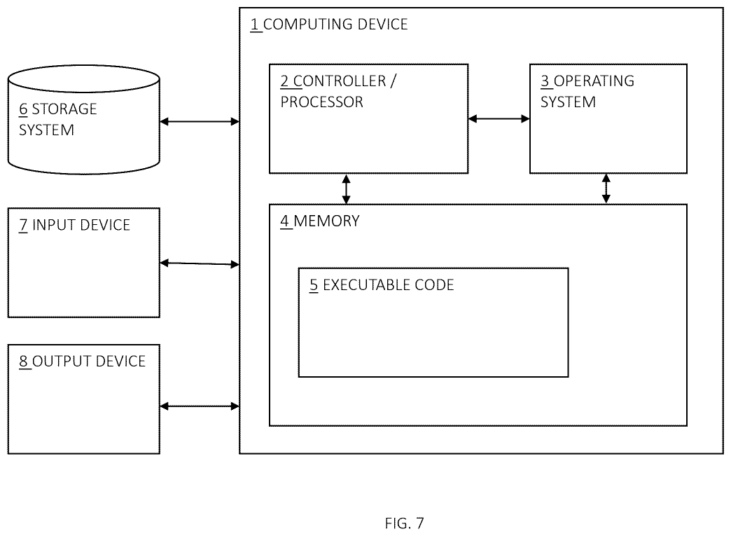

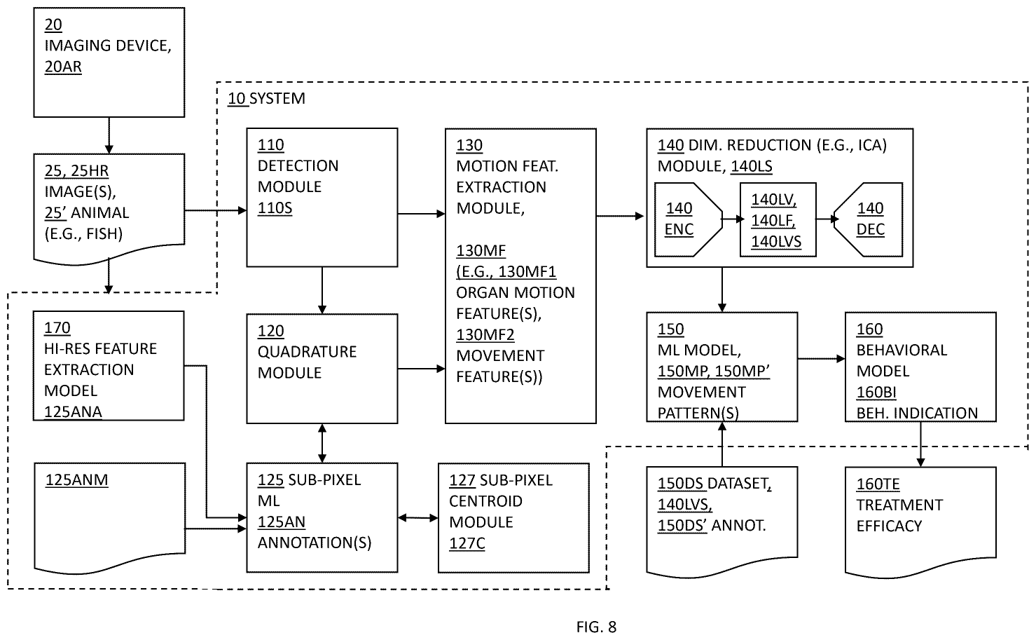

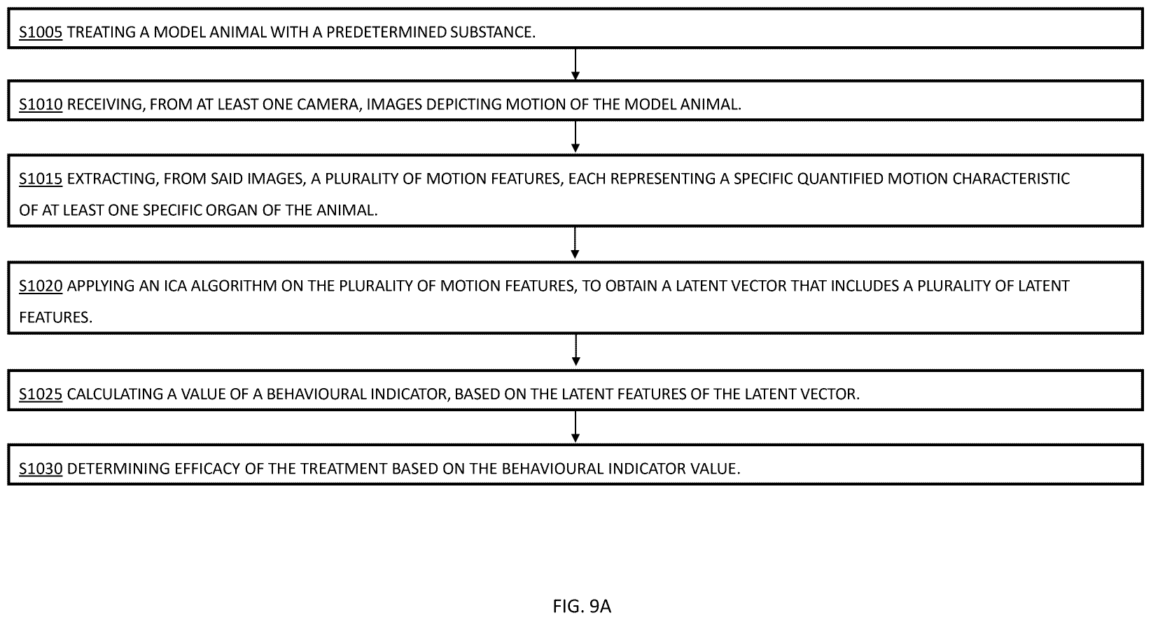

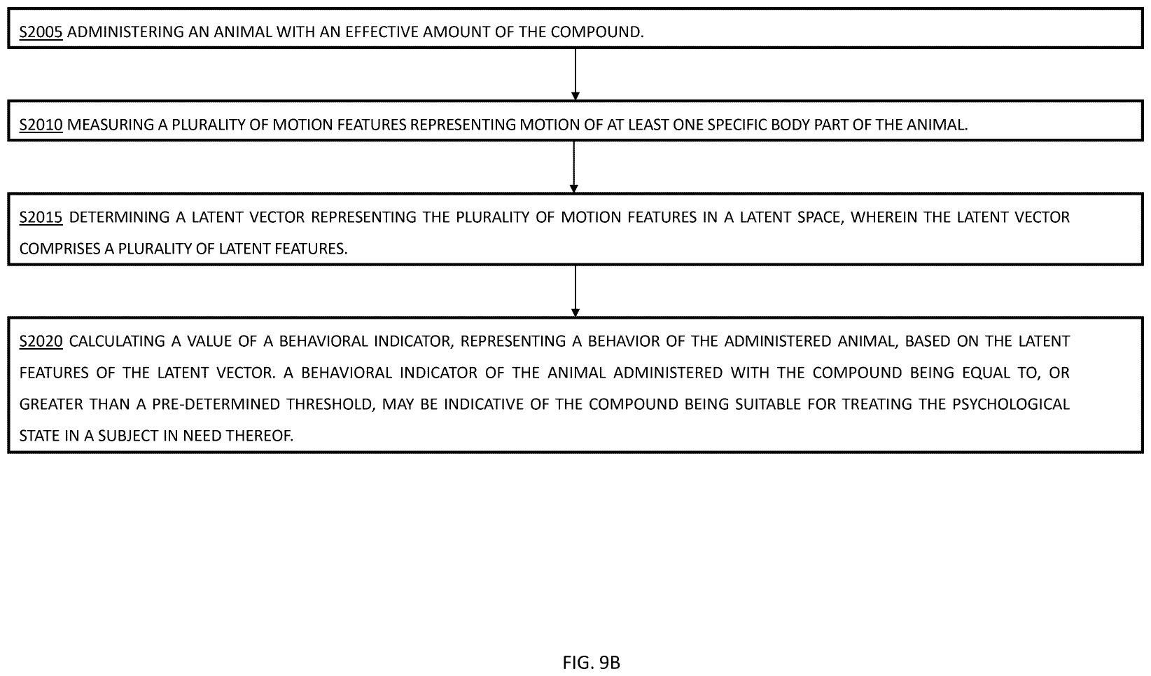

The left panel of FIG. 4D depicts latent space (e.g., independent component) analysis, which reveals the shift of swim patterns toward turn/escape behavior (latent feature 140LF ICA2) after cold stress. Pretreatment with psilocybin prevented such a shift. The same number (1200) of randomly selected swim events were plotted for each condition. Color saturations of dots and contour lines represent the local densities of all the collected data points.

The right panel of FIG. 4D depicts statistical analyses of the occurrences of turning/escape behaviors based on the latent feature 140LF ICA2 component, according to some embodiments of the invention. For the statistical analyses, Kernel density 2-sample test has been used. The inventors have included 4,216 (C), 3,833 (S) and 4,872 (P/S) swim episodes for the statistics of spontaneous exploration, and 3,312 (C), 2,713 (S) and 2,706 (P/S) swim episodes for the statistics of visually driven behavior. ***, p=1.8*10⁻³¹ (C vs S) and 3.0*10⁻⁸ (S vs P/S) during spontaneous exploration. ***, p=1.0*10⁻⁷ (C vs S) during visually driven swimming.

FIG. 4E depicts analyses of individual swim parameters, according to some embodiments of the invention. Acute cold stress exposure significantly increased tail frequency, tail angle and the number of tail motions, while such an effect was not observed in fish pretreated with psilocybin. Statistical tests used Tukey's post-hoc test after one-way ANOVA among different conditions during spontaneous explorations: **, p=0.0035 (tail frequency); *, p=0.032 (tail angle); *, p=0.046 (tail motions). Error bars represent standard deviations across tested fish. Error bars represent standard deviations across tested fish.

FIG. 4F depicts a summary of findings in the study, which are made possible through the implementation of the current invention. Psilocybin may act on subcortical structures that are evolutionarily conserved between fish and mammals. It has been determined that, psilocybin stimulates spontaneous behaviors and prevents stress-induced behavioral changes by creating an intermediate behavioral state between spontaneous exploration and visually driven swimming in larval zebrafish.

FIG. 4G shows representative swimming trajectories of the control and psilocybin-treated fish during a 5-minute spontaneous exploration in an environment with light and dark areas. The numbers of plotted swim events are shown at the bottom of the panel.

FIG. 4H shows the spatial distribution of swim events relative to the border between the light and dark areas in control (gray) and psilocybin-treated (black) fish based on analysis of 23,103 and 27,303 swim events from 30 control and 30 psilocybin-treated fish, respectively. Density plots, as well as binned histograms (9-mm intervals) from individual fish, were plotted. Error bars represent the standard error of the mean (s.e.m.) across the tested fish. **, p=0.0082 from kernel density 2-sample test between the control and psilocybin-treated fish.

FIG. 4I depicts swimming distance during spontaneous exploration and visually driven swimming after exposure to varying concentrations (25 mM, 50 mM and 100 mM) of sodium chloride in the water as hypertonic stress. N=14 fish for all conditions. We used Tukey's post-hoc test following one-way ANOVA analysis of spontaneous exploration among different conditions for statistics. **, p=0.049. Error bars represent standard deviations.

FIG. 4J depicts head centroid trajectories around the central part of the large arena after sham treatment (left) and hypertonic stress exposure (right) during visually driven swimming.

The left panel of FIG. 4K depicts latent space (independent component) analysis, which reveals the shift of swim patterns toward turn/escape behavior (ICA2) after hypertonic stress exposure. The same number (1200) of randomly selected swim events were plotted for each condition.

The right panel of FIG. 4K depicts statistical analyses of the occurrences of turning/escape behaviors based on latent feature 140LF ICA2 component. The inventors have used kernel density 2-sample test for statistics. The inventors have included 5,227 (control), 6,259 (25 mM), 5,514 (50 mM) and 5,046 (100 mM) swim episodes for the statistics of spontaneous exploration, and 4,730 (control), 3,322 (25 mM), 3,370 (50 mM) and 3,267 (100 mM) swim episodes for the statistics of visually driven behavior. ***, p=4.2*10⁻²⁸ (control vs 100 mM for spontaneous exploration); ***, p=4.9*10⁻⁵ (control vs 100 mM for visually driven swimming).

The left panel of FIG. 4L depicts latent feature (e.g., independent component) analysis of swim patterns after a “sham” treatment, where the fish was moved to a new dish containing a normal water (instead of salt water), stayed there for 10 minutes, and then was put back into a large dish of normal water for “recovery”, as obtained from 11 fish;

The central panel of FIG. 4L depicts hypertonic stress exposure data, obtained from 11 fish;

The right panel of FIG. 4L depicts psilocybin pretreatment and stress exposure data, obtained from 11 fish;

The left panel of FIG. 4M depicts the structure of Ketanserin and its affinities to binding targets;

The right panel of FIG. 4M depicts latent feature (e.g., independent component) analysis of swim patterns after the sham treatment, Ketanserin exposure and psilocybin pretreatment before Ketanserin exposure;

FIG. 5A depicts the chemical structure and dosage of ketamine (Ket).

FIG. 5B depicts Swimming distances during spontaneous exploration and optomotor response. N=12 (C), 14 (Ket), 10 (S) and 12 (Ket/S) fish. *, p=1.4*10-4 (C vs. S) and 1.7*10-3 (S vs. Ket/S) from Tukey's post-hoc test after one-way ANOVA for spontaneous swimming distance.

FIG. 5C shows swim patterns.

FIG. 5D shows the chemical structure and dosage of fluoxetine (Flx).

FIG. 5E presents swimming distances during spontaneous exploration and optomotor response.

FIG. 5F depicts swim patterns during optomotor response.

FIG. 5G presents a summary of the behavioral effects of tested antidepressants.

FIG. 5H presents a summary plot of the behavioral effect of tested antidepressants estimated from changes in independent components (IC1, IC2).

FIG. 6A is a scheme presenting another setup of neural activity imaging experiments, where fish is immobilized in an imaging chamber and motor signals from the tail are recorded using a pair of electrodes.

FIG. 6B is a behavioral paradigm (left) and visual gratings stop for 10 seconds during the spontaneous period (Spont.) and move forward for 10 seconds to induce optomotor response (OMR) during the OMR period. Right, trial-averaged swim patterns of control (N=6) and psilocybin-treated fish (N=7).

FIG. 6C shows the location of the dorsal raphe nucleus (DRN) in the zebrafish brain (left) and its image of expressing nuclear-localized calcium indicator (right). Scale bar, 20 μm).

FIG. 6D shows the spatial distributions of serotonergic neurons and GABAergic neurons in the DRN. Scale bar, 20 μm.

FIG. 6E shows the spatial distribution of neurons exhibiting higher activity during the spontaneous period (left) and the OMR period (right) in a representative fish. Differences in ΔF/F between these two task periods are shown for each neuron.

FIG. 6F shows (left) trial-averaged activity patterns of neurons activated during the spontaneous period (Class 1 neurons) and those activated during the OMR period (Class 2 neurons) from a representative fish; and fractions (right) of significantly active neurons during the spontaneous period (left bar of each two paired bars) and the OMR period (right bar of each two paired bars) in control and psilocybin-treated fish. N=6 and 7 for control and psilocybin-treated fish, respectively. *, p=0.046 for neurons activated during the spontaneous period by Wilcoxon's rank-sum test.

FIG. 6G is a low dimensional representation (left) of neural state dynamics in control fish and psilocybin-treated fish.

FIG. 7 is a block diagram, depicting a computing device which may be included in a system for determining efficacy of treatment according to some embodiments; and

FIG. 8 is a block diagram, depicting a system for determining efficacy of treatment according to some embodiments;

FIG. 9A is a flow diagram, depicting a method of determining efficacy of treatment according to some embodiments of the invention; and

FIG. 9B is a flow diagram, depicting a method of screening for a compound suitable for treating a psychological state in a subject in need thereof by at least one processor, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

The following table may be used as a reference or definition of terms used herein, for the reader's convenience:

TABLE 1

| | |
|---|---|
| Motion | The term “motion features” may be used herein to refer to one or more |
| feature | primary characteristics of an animal's motion, which may be extracted |
| | from one or more images (e.g., a movie), depicting the animal. In the |
| | non-limiting example where the depicted animal is a fish, motion |
| | features may include features of tail motion, features of head motion, |
| | features of fin, or limb motion, features of eye motion, features of heart |
| | motion, and features of traversal (e.g., swimming) intervals. |
| Body part, | The term “body part” may be used herein to refer to a portion or an |
| body points | organ of an animal as depicted in an image. In the non-limiting |
| | example where the depicted animal is a fish, a body part may include |
| | for example a head of the fish, a tail of the fish, an eye of the fish and a |
| | heart of the fish. The terms “point” or “body point” may be used herein |
| | interchangeably to refer to an exact position on the animal, or in a body |
| | part of the animal, as extracted from an image depicting the animal. |
| | For example, a body point of a fish may include location of a centroid |
| | point of the fish's head, in sub-pixel resolution, as elaborated herein. |
| Movement | The terms “movement patterns”, and “movement patterns classes” may |
| patterns, | be used herein interchangeably to refer to groups or types of |
| movement | movements of the depicted animal, extracted by analysis of the animals |
| patterns | motion features. In the non-limiting example of a fish, movement |
| classes | patterns may include, for example, short scooting, rapid long scooting, |
| | performance of routine turns, performance of C-turns, and a pattern of |
| | immobility. |
| Behavioral | The term “behavioral indicator” may be used herein to refer to a data |
| indicator, | element representing an extent to which a depicted animal presents a |
| behavioral | predefined behavior or behavioral state, and may be calculated based |
| state | on the animal's classification of movement patters. For example, a |
| | behavioral indicator of an animal may include one or more numerical |
| | scores representing levels of behavioral states such as anxiety of the |
| | animal, arousal of the animal (e.g., when chasing food), responsiveness |
| | of the animal to a visual stimulus, responsiveness of the animal to an |
| | odor stimulus, responsiveness of the animal to an acoustic stimulus, |
| | motoric disability of the animal, appetite of the animal, sleepiness of |
| | the animal, and the like. |
| Neural | The term Neural Network (NN) or Artificial Neural Network (ANN), |
| Network | e.g., a neural network implementing a Machine Learning (ML) or |
| (NN), | Artificial Intelligence (AI) function, may be used herein to refer to an |
| Artificial | information processing paradigm that may include nodes, referred to as |
| Neural | neurons, organized into layers, with links between the neurons. The |
| Network | links may transfer signals between neurons and may be associated with |
| (ANN), | weights. A NN may be configured or trained for a specific task, e.g., |
| Machine | pattern recognition or classification. Training a NN for the specific task |
| Learning | may involve adjusting these weights based on examples. Each neuron |
| (ML), | of an intermediate or last layer may receive an input signal, e.g., a |
| Artificial | weighted sum of output signals from other neurons, and may process |
| Intelligence | the input signal using a linear or nonlinear function (e.g., an activation |
| (AI) | function). The results of the input and intermediate layers may be |
| | transferred to other neurons and the results of the output layer may be |
| | provided as the output of the NN. Typically, the neurons and links |
| | within a NN are represented by mathematical constructs, such as |
| | activation functions and matrices of data elements and weights. At least |
| | one processor (e.g., processor 2 of FIG. 7) such as one or more CPUs or |
| | graphics processing units (GPUs), or a dedicated hardware device may |
| | perform the relevant calculations. |

Previous studies examining the effect of drug treatments on behavioral stress responses in zebrafish have generally focused on macroscopic parameters such as overall travel distance and environmental preference. The lack of body kinematics information in these studies makes it challenging to connect observed behavioral changes and underlying neural mechanisms.

A barrier to using high-speed behavioral tracking to study stress responses is that the small chambers typically used for such behavioral tracking can themselves incur confinement stress. Thus, precision approaches have yet to be explored for studying the effects of drug treatments on stress responses in model animals such as zebrafish.

To overcome this challenge, the inventors have developed a machine-learning-based approach that tracks body kinematics in a large, unconfining environment, and infers changes in behavioral states by stress exposure and drug (e.g., psilocybin) treatments. This approach enabled the inventors to disambiguate distinct behavioral states governing spontaneous exploration, visually driven rapid swimming, and irregular swim patterns after stress exposure.

It has been experimentally observed that acute drug (e.g., psilocybin) treatment of zebrafish facilitated rapid swimming in the absence of visual stimuli (stimulatory effect) and prevented occurrence of irregular swim patterns following stress exposure (anxiolytic effect). These behavioral effects parallel clinical observations and open new opportunities for studying how drugs such as serotonergic psychedelics impact neural dynamics in subcortical structures.

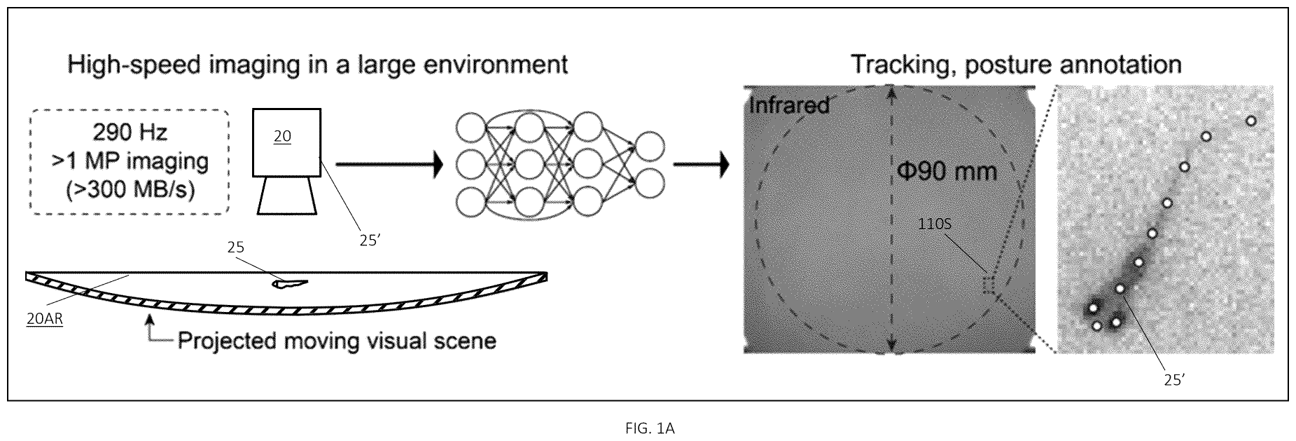

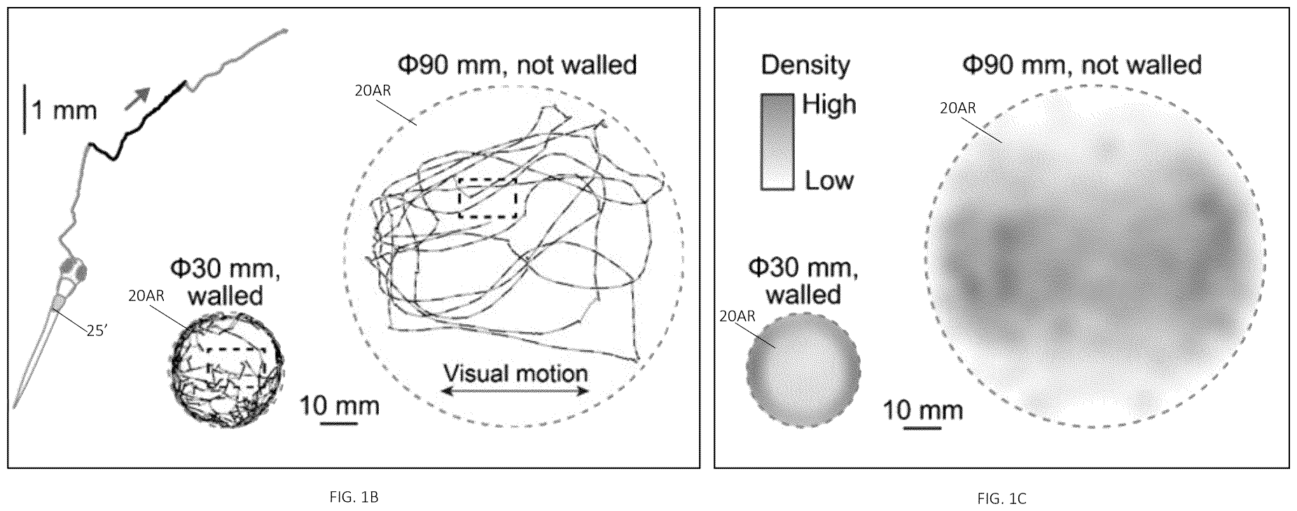

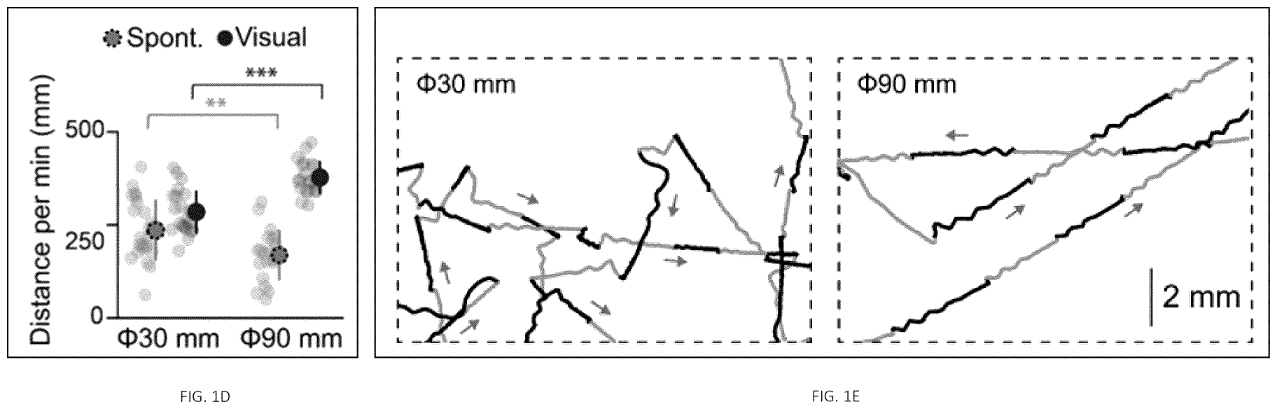

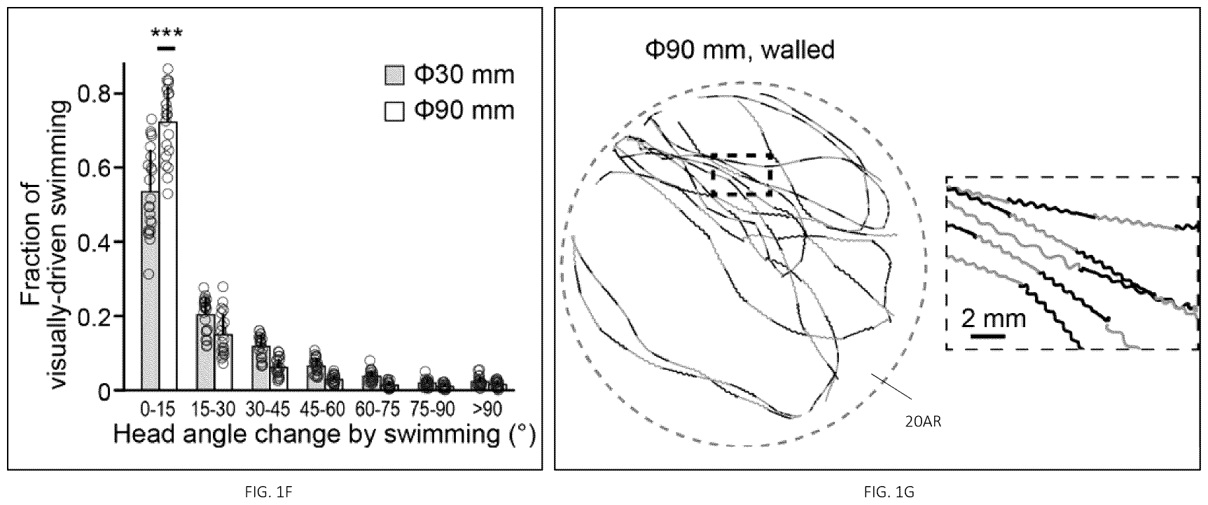

Reference is now made to FIG. 1A, which is a schematic diagram depicting an experimental setup, which may be used for evaluating behavior of an animal, according to some embodiments. As shown in FIG. 1A, the inventors have developed an experimental setup and a data processing pipeline that examines how innate behaviors of larval zebrafish, such as spontaneous exploration and optomotor response, change after drug treatments and stress exposure. This setup tracks the precise body kinematics of a single fish in an environment that is large (e.g., 90 mm) compared to the length of larval zebrafish (˜4 mm) at a high-resolution (e.g., higher than 1100×1100 pixels) and high-speed (e.g., 290 Hz). Data analysis pipelines further processed the images to identify the fish loci and body postures by using a deep neural network.

Reference is now made to FIGS. 1B-1F, which are diagrams depicting aspects of recorded movement of the animal, according to some embodiments.

As shown in FIG. 1B, head centroid trajectories in a small, walled environment (30 mm) and a large, unwalled environment (90 mm). Different swim episodes can be visually separated in the depicted trajectories. Fish behavior was recorded at infrared wavelengths for 15 minutes while visual gratings stopped for 10 seconds (spontaneous swimming) and moved for 10 seconds (visually driven behavior) in cycles.

FIG. 1C presents distributions of head centroids during experiments across tested fish, N=22 and N=20 fish for the small and large dishes, respectively. The inventors have tested a single fish per experiment to exclude the effect of social dynamics throughout this study. The large size of the imaged arena resulted in lower pixel resolution relative to previous body kinematics studies. Therefore, data processing was required to track head trajectories and tail kinematics at sub-pixel resolution, i.e., at spatial scales smaller than the pixel size enabled by prior knowledge of the shape of the animal. As shown in FIG. 1C, the inventors have achieved localization accuracy at around 25 m for the head trajectory.

In FIG. 1D, swimming distance per minute during spontaneous exploration and visually induced swimming were recorded. Small circles represent individual fish. Using the setup of FIG. 1A, the inventors have examined behavioral impacts of the size of behavioral arenas by comparing swimming trajectories between those in a small, walled arena (e.g., 30 mm) and those in a large unwalled arena (e.g., 90 mm). Zebrafish typically swam near the wall in the small arena due to their innate preferences called thigmotaxis. In a large arena, on the contrary, they explored widely (FIGS. 1B, 1C) and swam longer distances during visual stimulus motion (FIG. 1D). As shown in FIG. 1D, spontaneous swimming distance was longer in the small arena, indicating a stimulatory effect of confinement stress.

As shown in FIG. 1E, expanded head centroid trajectories from the outlined central parts of the small arena (left) and the large arena (right) from (B). The large environment facilitates straight swim patterns.

The inventors have also observed notable differences in swim trajectories around the central area of the dish. Fish showed frequent turning in the small arena even when they were not near the wall, potentially due to confinement stress, whereas they swam mostly in straight lines in the large arena (FIG. 1F). Fish also showed straight swim patterns in a different large arena with a boundary wall (FIG. 1G). These observations indicate that the size of the behavioral arena significantly impacts the swim patterns of larval zebrafish.

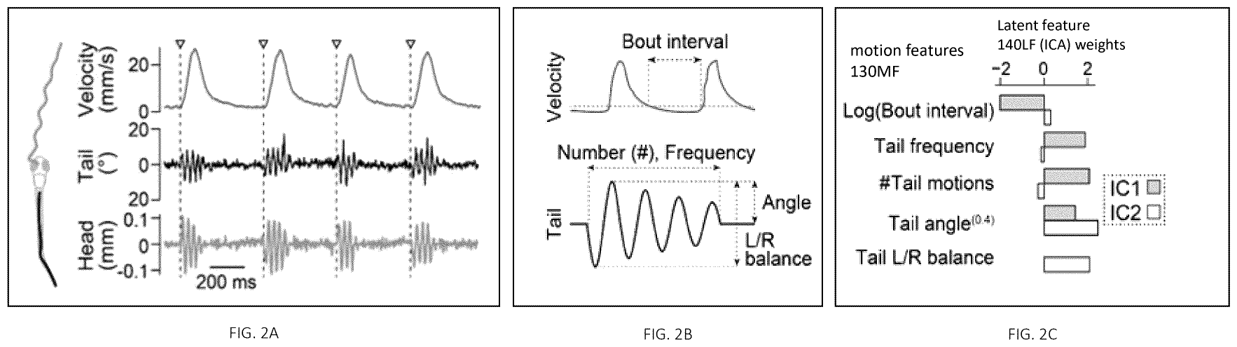

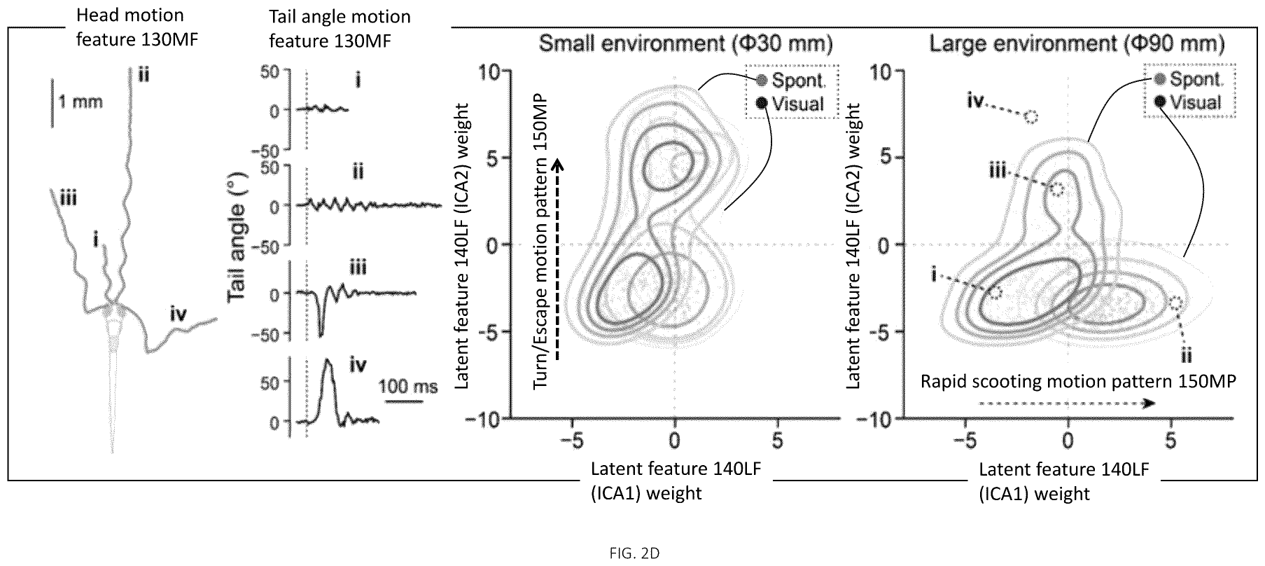

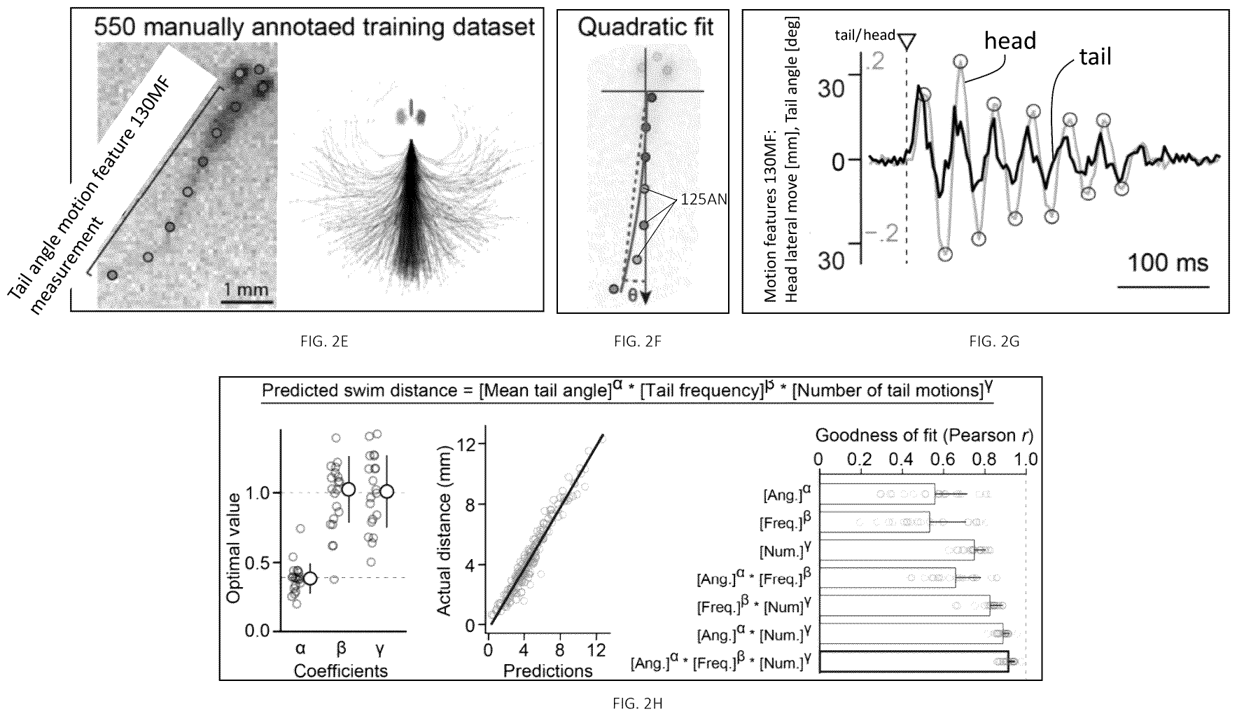

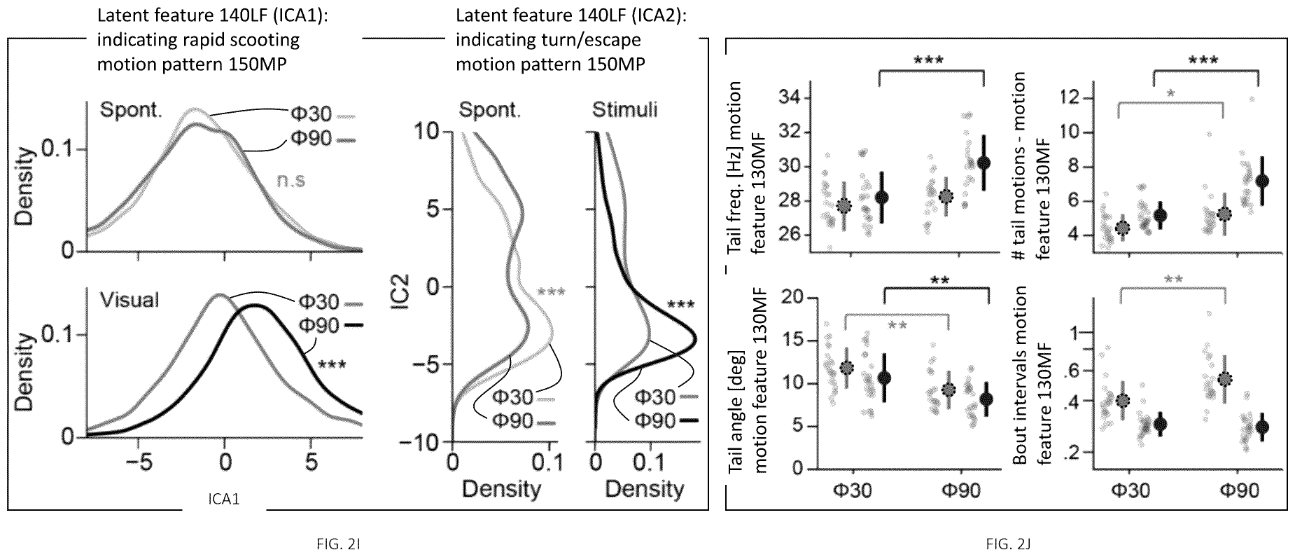

Reference is now made to FIGS. 2A-2D, and supplementary FIGS. 2E-2J, which depict stages in analyzing motion features of depicted animals, according to some embodiments of the invention.

FIG. 2A depicts swim velocities, tail motion, and head centroid motions during multiple swim episodes. Triangles represent the onset of individual swim episodes. The inventors have quantified tail motions (e.g., as depicted in FIG. 2A) using a deep neural network, as further described in detail herein.

FIG. 2E depicts annotations of point across an animals body, according to some embodiments. On the left panel, annotation of 10 points are shown along the body of a depicted zebra fish. On the right panel, distribution of body parts in 550 manually annotated training datasets. During development, the inventors applied manual annotations using fish images from various orientations. These annotations were aligned for the visualization of this panel. Training images were selected to balance various tail angles on the left and right sides.

In one example of implementation, the inventors have trained the Deep Neural Network (DNN) over 550 images, such as depicted in FIG. 2E, where points or parts of the animal's body were manually annotated. This annotation included ten body parts, such as the eyes, nostril, body trunk, and six points along the tail.

FIG. 2F depicts quantification of tail angle θ, by fitting a quadratic function to annotated points along the tail. As shown in FIG. 2F. The inventors have fitted quadratic curves to the identified points along the tail to quantify tail motions.

FIG. 2B depicts motion features (e.g., features of tail motion) and features of traversal (e.g., swimming features, or swim parameters), that may be obtained by embodiments of the invention, for reduced dimensionality analysis, e.g., Independent Component Analysis (ICA), as elaborated herein.

FIG. 2G presents an overlay of tail motions and head centroid motions during a representative swim episode. Peaks of head centroid motions as detected by embodiments of the invention are marked by circles. These peaks were used as a reference to quantify tail motions. As shown in FIG. 2G, lateral movements of the head centroid are always synchronized with the tail movements. Therefore, embodiments of the invention may use head centroid position as a reference for extracting the tail motion features of FIG. 2B. Additionally, embodiments of the invention may validate the accuracy of quantified tail movements by examining how well tail parameters can predict the swim distance for each swim event.

FIG. 2H presents validation of accuracy of tail angle quantification by predicting swimming distance based on tail motion parameters. On the left panel, parameters of a multiplicative prediction model were optimized by using an optimizer. On the central panel, the resulting model shows high correlations to swimming distance. The right panel presents quantification of the prediction accuracy tested in a large environment. Error bars represent standard deviations. The full model, presented by the bottom bar, demonstrated an accuracy of Pearson correlation coefficient r=0.89±0.036 across 20 fish.

As shown in FIG. 2H, a prediction model based on extracted tail parameters yielded a Pearson correlation coefficient of 0.89±0.036 across 20 fish, indicating a highly accurate extraction of tail motion parameters.

FIG. 2C depicts an example of weights for two latent features, also referred to herein as ICA components. A first latent feature, or ICA component, denoted herein component #1 or ICA1 (top bar of each two paired bars in FIG. 2C) and a second latent feature, or ICA component, denoted herein component #2 or ICA2 (bottom bar of each two paired bars in FIG. 2C).

FIG. 2H represents validation of accuracy of tail angle quantification by predicting swimming distance based on tail motion parameters, by some embodiments of the invention. On the left pane, parameters of a multiplicative prediction model were optimized by using an optimizer. On the center pane, the resulting model shows high correlations to swimming distance. On the right pane, quantification of the prediction accuracy tested in a large environment. The full model, shown at the bottom, has an accuracy of Pearson correlation coefficient r=0.89±0.036 across 20 fish. Error bars represent standard deviations.

The inventors have used tail angle as a power of 0.4 because it best correlated with the swim distance as shown herein (e.g., in FIG. 2H). Traversal features (also referred to herein as swim parameters) and motion features (also referred to herein as motion parameters) such as tail motion parameters may be Z-scored individually before performing ICA.

During development, the inventors have examined how arena sizes affect latent behavioral states by using latent features 140LF (ICA) of various, separate parameters of swimming, and/or motion features 130MF. Five such motion features 130MF are presented in FIG. 2C, and include: frequencies of tail motions, angles of tail motions, the number of tail motions, the balance of tail motions between left and right sides, and intervals between swim episodes.

It may be appreciated that such dimensionality reduction analysis, based on multiple parameters yields more robust estimates of latent behavioral states. The inventors have applied this analysis to swim episodes in the central part of small and large arenas, to identify latent feature 140LF (ICA) representation of motion features 130MF.

In a non-limiting example of implementation, the inventors identified the first two independent latent features 140LF or components, denoted herein ICA1 and ICA2, in an unbiased manner. These components enabled the inventors to map various types of movement patterns or traversal patterns into different loci on a low-dimensional (e.g., ICA) latent space 140LS.

FIG. 2D represents scatter density plots of various movement patterns or traversal patterns 150MP (or in this case—swim patterns 150MP) in the latent space 140LS (also referred to as ICA space 140LS), revealing enriched repertoires of swimming in a large environment.

On the left pane of FIG. 2D, motion features 130MF of head centroid trajectories and tail motions of four representative motion patterns (swim patterns) 150MP are presented.

On the right pane of FIG. 2D, scatter density plots of motion patterns (swim patterns) 150MP in the small and large environment, during spontaneous exploration and visually induced swimming are presented in the latent (ICA) space 140LS.

The same number (1200) of randomly selected swim events from N=22 and N=20 fish for the small and large dishes, respectively, were plotted for each condition. Color saturations of dots and contour lines represent the local densities of all the collected data points. The loci of four representative swim patterns 150MP on the left are marked in black circles. Larger environment (e.g., 90 mm arena 20AR) facilitated rapid long scooting (ICA1) during visually driven swimming and fewer turnings/escapes (ICA2) during both spontaneous and visually driven swimming.

FIG. 2I presents an example of statistical analyses of latent features 140LF (latent components ICA1 and ICA2), performed by the inventors, using the same set of fish as in FIGS. 1A-1G, by using kernel density 2-sample test. The inventors included 2,635 (small arena 20AR) and 5,233 (large arena 20AR) swim episodes for the statistics of spontaneous exploration and 1,466 (small arena 20AR) and 5,360 (large arena 20AR) swim episodes for the statistics of visually driven swimming. ICA1: n.s. (not significant), p=0.14; ***, p=6.0*10-49. ICA2: ***, p=1.4*10-12 and 1.1*10-4 for spontaneous and visually driven swimming, respectively.

In the non-limiting example, where the examined animal was a fish, the swim patterns 150MP may include, for example: (i) short scooting, (ii) rapid long scooting, (iii) routine turns, and (iv) C-turns. As shown in FIG. 2D, latent feature 140LF ICA1 separated short scooting during spontaneous exploration and long rapid scooting during visual stimulus motion. Latent feature 140LF ICA2 separated scooting and turning/escape behaviors. In other words, this mapping method showed a clear separation of motion (swim) patterns 150MP during visually driven swimming from those during spontaneous exploration in the large arena. Long rapid scooting 150MP (identified via ICA1) was dominant during visual stimulus motion in the large arena 20AR, whereas turning and escape motion patterns 150MP (identified via ICA2) were more dominant in the small arena 20AR, as shown in FIGS. 2D and 2I.

FIG. 2J presents an example of statistical analyses of individual traversal motion features (also referred to as swim features, or swim parameters), according to some embodiments of the invention. The large arena facilitated significantly higher frequencies, more numbers of tail motions and smaller tail angles during visually driven swimming, while it elongated bout intervals during spontaneous exploration. P values are from a 2-sample t-test between N=22 and N=20 fish for the small and large dishes, respectively. ***, p=2.3*10-4 (frequency); *, p=0.021, ***, p=2.8*10⁻⁶ (motions); ** for spontaneous exploration, p=1.4*10-3, ** for visually driven swimming, p=3.2*10-3 (angle); **, p=4.1*10-3 (interval). Error bars represent standard deviations across tested fish.

As shown by FIG. 2J, at the individual parameter level, the large arena allowed higher tail frequency and more tail motions per bout during visual stimulus motion and longer bout intervals during spontaneous exploration, consistent with the above observation of swim trajectories depicted in FIGS. 1B and 1C. These body kinematics analyses demonstrate that a large arena, which is more than 20 times the body length of larval zebrafish, is essential for evaluating the full extent of swimming repertoires while minimizing confinement-induced turning/escape behaviors.

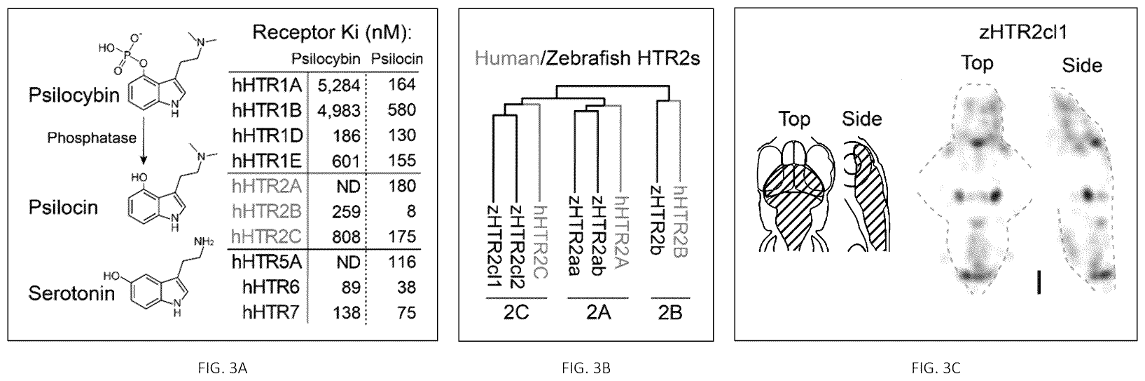

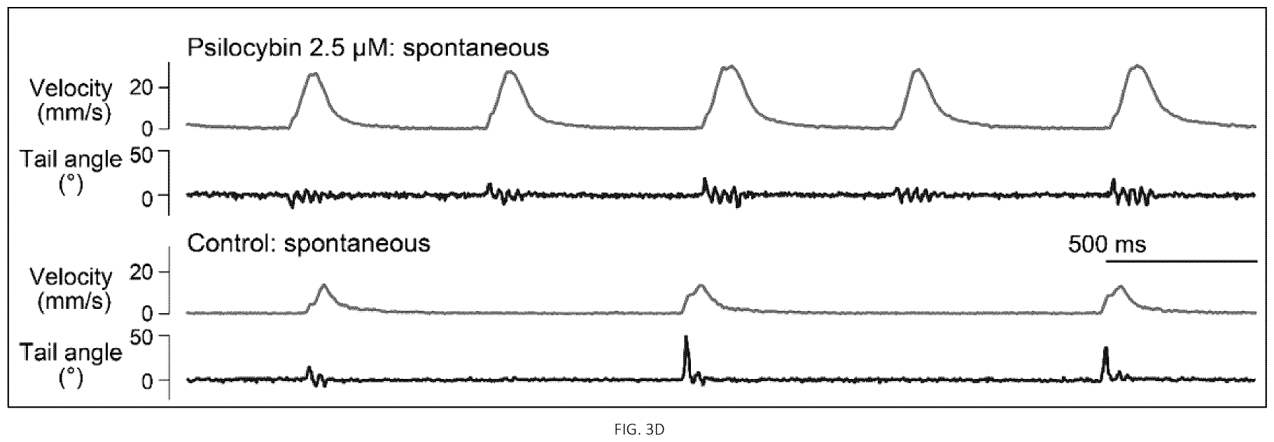

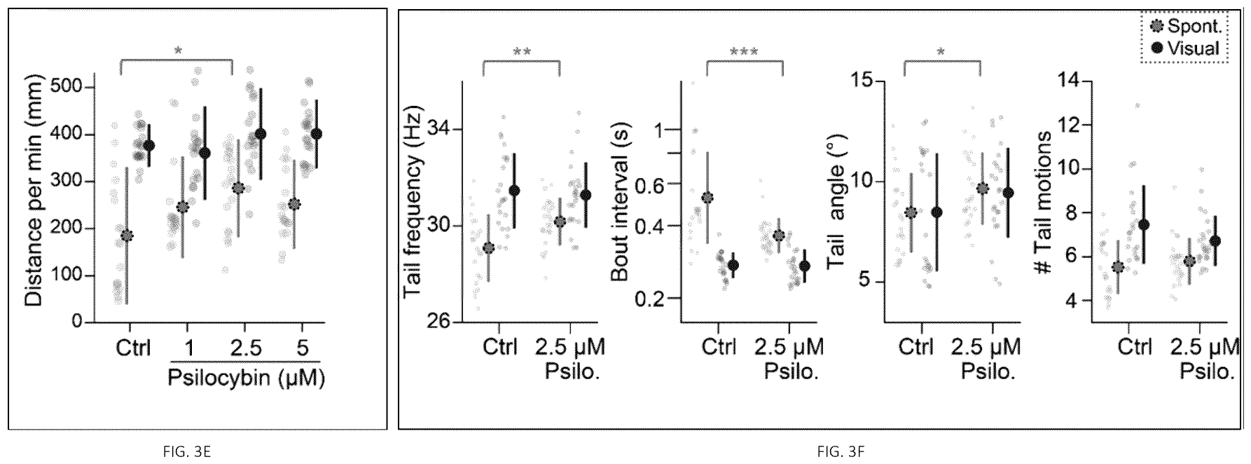

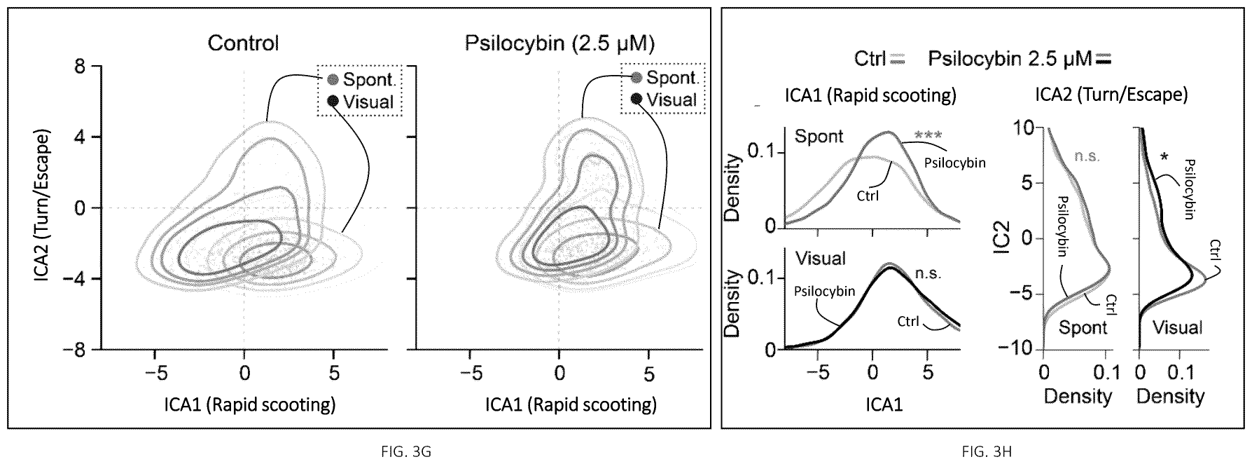

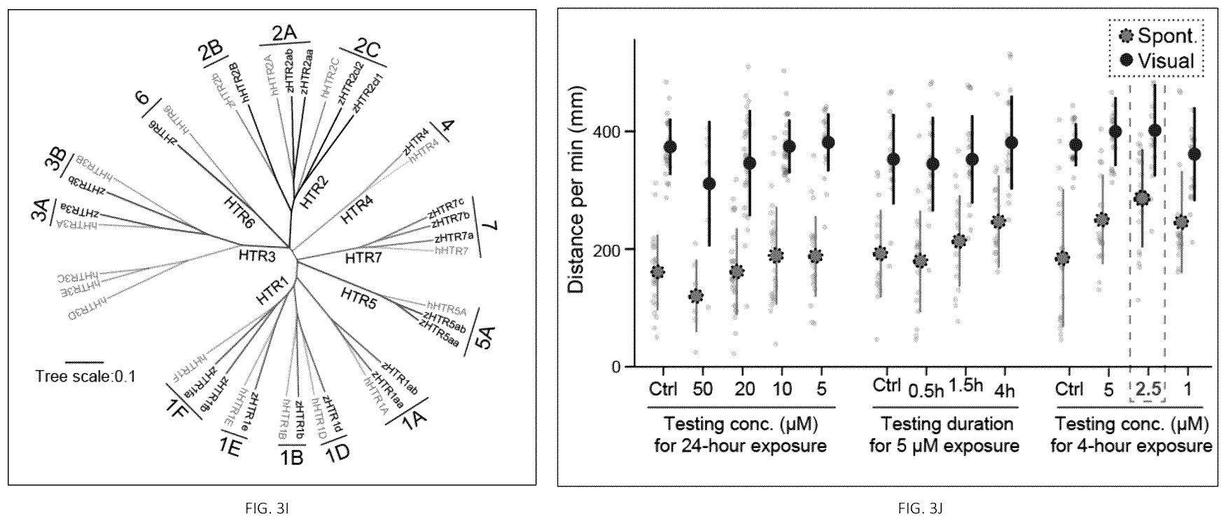

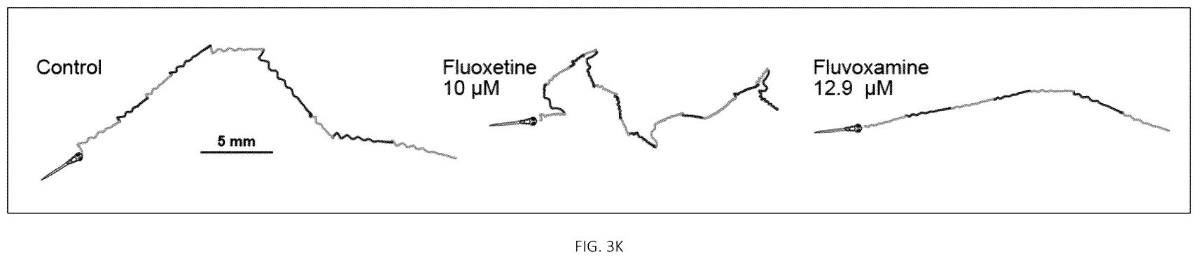

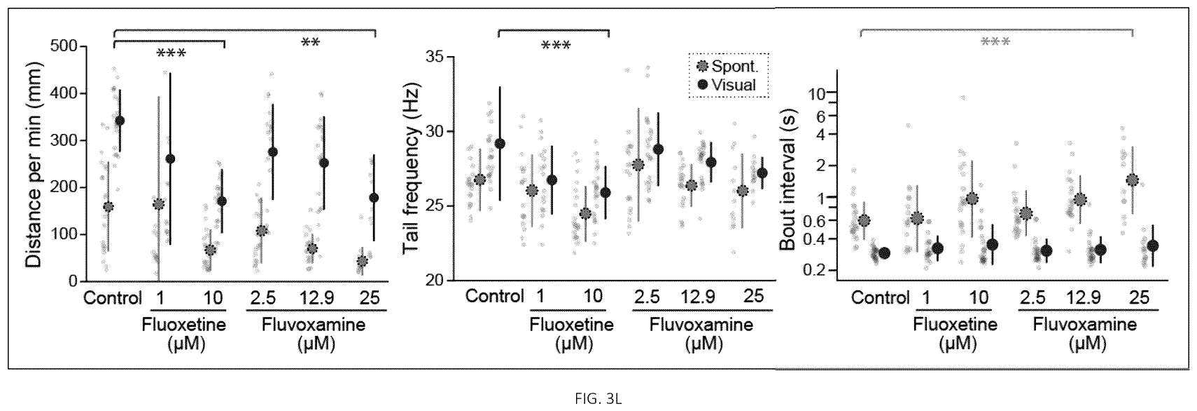

FIGS. 3A-3H represent aspects of effect of a predetermined treatment (e.g., a drug of interest, on motion features and movement patterns of the examined animal, according to some embodiments of the invention.

As shown in FIG. 3A, the inventors have tested the effects of psilocybin treatment on spontaneous exploration and visually driven swimming by using machine learning methodologies, as elaborated herein.

As known in the art, Psilocybin and its metabolite psilocin act as agonists for serotonin receptors. Upon ingestion, psilocybin may convert to psilocin by the action of endogenous phosphatases. Psilocin may cross the blood-brain barrier and may have stronger affinities to serotonin receptors. Psilocin has affinities to multiple types of serotonin receptors, including inhibitory HTR1 receptors and excitatory HTR2 receptors. Serotonin receptors in zebrafish are highly similar to those in humans and include major types from HTR1 to HTR7 and their subtypes.

FIG. 3B depicts unbiased homology analysis of protein sequences revealed conserved subclasses of type 2 serotonin receptors between zebrafish and humans. FIG. 3I depicts unbiased homology analyses of protein sequences of all serotonin receptors revealed conserved major types and subtypes between zebrafish and humans. As shown in FIGS. 3B and 3I, unbiased homology analysis of protein sequences showed robust co-clustering of human and zebrafish serotonin receptors down to subtypes such as HTR2A, 2B, and 2C.

FIG. 3C depicts average expression mapping of HTR2cl1 gene across 5 zebrafish brains obtained by using RNA fluorescence in situ hybridization. Scale bar, 100 μm.

As shown in FIG. 3C, the inventors confirmed the high expression of zebrafish HTR2 receptors in the brain. Therefore, it is reasonable to hypothesize that psilocybin and its metabolite psilocin have behavioral effects on larval zebrafish.

FIG. 3J depicts the effect of psilocybin treatment on swimming distances during spontaneous exploration and visually driven swimming. We tested various durations and concentrations (conc.) of psilocybin exposure. Data from the same set of experimental batches are clustered together with their individual control data. Numbers of fish (left to right): N=22 (Ctrl), 6 (50 μM), 25 (20 μM), 26 (10 μM), 21 (5 μM), 17 (Ctrl), 18 (0.5 h), 18 (1.5 h), 18 (4 h), 18 (Ctrl), 18 (5 μM), 18 (2.5 μM) and 18 (1 μM). The data on the right (4-hour exposure) is the same as those shown in FIG. 3E. The condition in the dashed box (2.5 μM, 4 h) had the strongest impact on spontaneous exploration and was used for experiments in FIGS. 3 and 4.

As shown in FIG. 3J, the inventors have found that acute, short bath pretreatment with psilocybin (2.5 μM, 4 h) in larval zebrafish had stimulatory effects on spontaneous exploration. The inventors determined this pretreatment protocol after testing dosages between 1 uM and 50 uM and durations between 30 minutes to 24 hours. The concentration of 2.5 uM amounts to a slightly higher dosage (0.71 mg/kg) compared to the clinical dosage in humans (0.6 mg/kg). This optimal duration of 4 h is consistent with the time course of passive diffusion of a drug with similar molecular weight into the brain of larval zebrafish.

The inventors have observed the reduction of such effects at higher concentrations and longer durations, indicating that the action of psilocybin becomes saturated at this relatively low concentration compared to serotonin-selective reuptake inhibitors (see below). This saturation effect is consistent with clinical observations in human subjects.

FIG. 3D depicts evocation, by Psilocybin, of rapid scooting behaviors during spontaneous exploration.

FIG. 3E depicts another effect of Psilocybin at a concentration of 2.5 μM. In this concentration, Psilocybin significantly enhances swimming distances during spontaneous exploration but not visually driven swimming. N=18 fish for each condition. *, p=0.011 from Tukey's post-hoc test after one-way ANOVA detected a significant difference (F=3.6) among groups for spontaneous swimming distance.

FIG. 3F depicts another effect of Psilocybin, which significantly enhanced tail frequency, shortened bout intervals, and slightly enhanced tail angles during spontaneous swimming. N=22 and 23 fish for control and 2.5 μM conditions, respectively. P values are from a 2-sample t-test between groups. **, p=0.0051 (frequency); ***, p=8.6*10-4 (interval); *, p=0.044 (angle).

After pretreatment with psilocybin, fish typically swam with shorter intervals with faster velocities (FIG. 3D), resulting in enhanced swim distance during spontaneous exploration (FIG. 3E). They showed significantly enhanced tail frequencies and shorter intervals between swim bouts similar to those observed during visual stimulus motion (FIG. 3F).

The inventors have not seen noticeable changes in these parameters during visual stimulus motion, indicating that psilocybin's effect is limited to spontaneous exploration in this implementation example.

FIG. 3G shows that Independent Component Analysis (ICA) may reveal a shift of spontaneous swim patterns toward the distribution of visually driven swim patterns. The same number (1200) of randomly selected swim events were plotted for each condition. Color saturations of dots and contour lines represent local densities of all the collected data points.

FIG. 3H shows that Psilocybin may significantly enhance rapid scooting (ICA1) during spontaneous exploration, while it does not cause a significant increase in turning/escape behaviors (ICA2). Statistical analyses of ICA1 were performed using the same set of fish in FIG. 1 by using kernel density 2-sample test. We included 6,128 (control) and 9,395 (2.5 μM) swim episodes for the statistics of spontaneous exploration and 6,413 (control) and 6,427 (2.5 μM) swim episodes for the statistics of visually-driven behavior. ***, p=3.3*10⁻⁶⁵ and *, p=0.013 between the control group and those after exposure to 2.5 μM psilocybin. n.s. (not significant), (p>0.05). Error bars represent standard deviations across tested fish.

Independent Component Analysis of swim parameters also confirmed the observation of FIG. 3G. As shown in FIG. 3H, the inventors observed a significant shift in spontaneous swim patterns toward the direction of visually driven rapid scooting along the axis of ICAL. These results indicate that psilocybin stimulates swim patterns in a partially similar manner to visual stimuli.

FIG. 3K presents swim trajectories of control, fluoxetine-treated, and fluvoxamine-treated fish during visually driven behaviors, according to some embodiments. The effect of acute exposure to Psilocybin was different from that of Serotonin-Selective Reuptake Inhibitors (SSRIs) that block serotonin reuptake and increase serotonin concentration at synapses. Full therapeutic effects of SSRIs do not occur during acute dosage in humans, and some studies showed elevated anxiety levels during the first few weeks of SSRI treatment. Consistently with previous reports in zebrafish, the inventors observed that pretreatments with fluoxetine and fluvoxamine have suppressive effects on swim patterns.

As shown in FIG. 3K, while fluoxetine caused noticeable distortions in the swim patterns and low-frequency tail motions, fluvoxamine may decrease amplitude of tail motions.

FIG. 3L depicts swimming distance per minute (left pane), tail frequency (center pane) and average tail angle (right pane) of control (N=21), fluoxetine-treated (N=17 for 1 μM, N=20 for 10 μM) and fluvoxamine-treated fish (N=20 for 2.5 μM, N=20 for 12.9 μM, N=12 for 25 μM), according to some embodiments. P values are from Tukey's post-hoc test after one-way ANOVA analysis. ***, p=3.8*10-5; **, p=1.1*10-3 (distance per minute during visually driven swimming); ***, p=6.4*10-4 (tail frequency during visually driven swimming); ***, p=2.5*10-4 (bout intervals during spontaneous exploration). Error bars represent standard deviations.

As shown in FIG. 3L, swimming distances decreased linearly with the dosage for both spontaneous exploration and visually driven swimming, indicating that SSRIs suppress motor circuits in the brain regardless of external stimuli. These differences in behavioral effects between psilocybin and SSRIs suggest that psilocybin's stimulatory effects may occur from its selective affinities to a subset of serotonin receptors (e.g., depicted in FIG. 3A).

FIG. 4A is a schematic diagram depicting behavioral paradigms for psilocybin treatment and acute cold stress exposure.

As known in the art, acute administration of psilocybin has anxiolytic effects in humans. The inventors have thus tested whether acute stress exposure causes changes in fish's swim patterns in our setup and whether psilocybin can prevent such stress-induced behavioral changes. Various environmental stressors have been tested in larval zebrafish that trigger cortisol increase, including hypertonic water, acids, mechanical disturbance, social isolation, and heating or cooling shock. As shown in FIG. 4A, the inventors used an acute cold shock protocol that rapidly lowers the temperature by 10 degrees (e.g., from 28° C. to 18° C.) as it is least likely to cause physiological stress from lasting changes in tissue integrities, protein folding, and ionic balance in the body.

As shown in FIG. 4I-E, the inventors also tested the effect of hypertonic stress for comparison. The inventors pre-treated fish with psilocybin with the most effective concentration for enhancing spontaneous exploration (2.5 μM, also refer to FIG. 3), exposed them to stressors for 5 minutes, recovered them at a normal temperature, and tested their spontaneous exploration and visually driven swimming (FIG. 4A).

As shown in FIGS. 4B and 4I, exposure to both cold and hypertonic stressors increased the swimming distance, which is consistent with the previous studies, and demonstrates the robustness of the stress protocol.

As shown in FIG. 4C, the inventors found that acute stress exposure caused “zig-zag” swimming patterns compared to the control. During visual stimulus motion, the control fish shows straight trajectories in our large arena (also refer to FIG. 1F). Such patterns changed into zigzag patterns, as each bout started from a sharp turning of the head and large tail undulation to one side (pattern [iii] in FIG. 2D), instead of smooth scooting (patterns [i] or [ii] in FIG. 2D). This type of change was also observed after exposure to hypertonic stress (also refer to FIG. 4J), suggesting that the emergence of zig-zag swim patterns, as compared to normal smooth straight patterns, can be a robust indicator of stress-induced behavioral changes in larval zebrafish.

Inventors have found that pre-treatment with psilocybin prevented stress-induced changes in swim patterns. As shown in FIG. 4C, Psilocybin-pretreated fish exhibited straight swim patterns even after the stress exposure.

As shown in FIG. 4D, this prevention of stress-induced behavioral changes was also evident in the ICA analysis along the axis of ICA2, which represents occurrences of escape/turning behavior. Acute cold stress significantly elevated distributions along the ICA2 axis. The pretreatment with psilocybin significantly diminished the increased occurrence of turning/escape behavior after cold stress.

As shown in FIG. 4E such preventative effect was also evident in individual tail kinematics, such as frequencies and angles of tail motions. Notably, psilocybin pretreatment did not prevent the stress-induced shift of behavioral states along the ICA1 axis, which represents occurrences of rapid scooting behavior (FIG. 4D). This shift along the ICA1 axis was found to be consistent with the effect of psilocybin pretreatment per se (FIG. 3G). These results suggest that the stimulatory effect of psilocybin may prevent the stress-induced occurrence of escape/turn behavior at the same time.

It was also examined whether psilocybin mitigates the innate anxiety response of larval zebrafish by measuring the dark avoidance behavior (FIG. 4G). The inventors found that psilocybin-treated fish explore the darker side significantly more often than the control fish (FIG. 4H). This result indicates that psilocybin can ameliorate both innate and externally induced anxiety responses.

As shown in FIG. 4L, the inventors did not observe similar preventative effects of psilocybin for behavioral changes induced by hypertonic stress. This discrepancy is potentially due to the difference between central, anxiety-like stress and physiological stress for larval zebrafish, and may indicate that psilocybin's anxiolytic effects may result from preventing the occurrence of anxiety-related neural dynamics rather than from inducing straight swim patterns at the motor circuit level.

The behavioral effects of other antidepressants was investigated and compared with those of psilocybin. Initially, the effect of ketamine was tested. Ketamine is a fast-acting antidepressant that can also suppress stress-induced behavioral changes in zebrafish. Sub-anesthetic concentration (30 μM) were used and bath application were performed for 30 minutes before the cold shock (FIG. 5A). Unlike psilocybin, ketamine did not increase spontaneous swimming distance and reversed the stress-induced increase in swimming distance (FIG. 5B). However, ketamine prevented the emergence of zig-zag swim patterns after cold shock and recovered straight swim patterns (FIG. 5C). In the ICA analysis, ketamine significantly reversed the stress-induced shift along the IC2 axis. These behavioral effects of ketamine demonstrate that the behavioral analysis based on ICA of body kinematics as presented herein generalizes to the effect of other anxiolytic drugs and that psilocybin has a similar acute anxiolytic effect as ketamine.

The effect of fluoxetine was also tested in the cold shock paradigm, using a concentration of 4.6 μM from previous zebrafish studies, and the effect of bath application was examined for 24 hours before the cold shock (FIG. 5D). Similar to ketamine, fluoxetine did not increase spontaneous swimming distance and reversed the stress-induced increase in swimming distance (FIG. 5E). However, fluoxetine had mixed effects on body kinematics after stress exposure. It partially reversed and partially exacerbated shifts of behavioral states along the IC2 axis (FIG. 5F). These mixed effects may be due to the induction of distorted swim patterns by fluoxetine that were observed (FIG. 5F, FIG. S3C) but also may reflect its lack of acute anxiolytic effects in humans.

The inventors classified the effects of psilocybin, ketamine and fluoxetine based on two behavioral measures: changes in swimming distances (FIG. 5G) and the ICA analysis of body kinematics (FIG. 5H). In the first type of classification (FIG. 5G), the action of tested drugs was classified based solely on swimming distances. Stimulatory/suppressive effects were defined as changes in spontaneous swimming distance in unstressed fish by the application of the drugs compared to the control condition. Anxiolytic effects were calculated based on how much the drugs could revert the increase of swimming distance induced by stress exposure. In the second type of classification (FIG. 5H), two dimensions identified by the ICA analysis of body kinematics were used. Stimulatory/suppressive effects were defined as shifts along the IC1 axis in unstressed fish by the application of the drugs compared to the control condition. Anxiolytic effects were calculated based on how much the drugs could revert the changes along the IC2 axis induced by stress exposure.

The results of these two types of classifications were compared. Their stimulatory/suppressive effects on basal behavior are consistent between these two measures, where fluoxetine is suppressive and psilocybin is stimulatory. However, their effects on stress-induced behavioral changes were different. The reversal of the stress-induced increases in swim distances (FIG. 5G) only occurred with ketamine and fluoxetine. In contrast, the reversal of the stress-induced changes in body kinematics (FIG. 5H) only occurred with ketamine and psilocybin. These results demonstrate that the use of a single behavioral indicator such as swimming distance may confound stimulatory/suppressive effects and anxiolytic effects of antidepressants and that dimensionality reduction analysis based on multiple body kinematic parameters provides a more accurate measure of their anxiolytic effects.

The inventors further examined the involvement of HTR2 receptors in stress-induced behavioral changes by using Ketanserin, an HTR2 receptor antagonist which also inhibits monoamine transporters and histamine receptors. Ketanserin has acute anxiogenic effects in adult zebrafish and rodents. As shown in FIG. 4M, the inventors found that bath application of Ketanserin shifted behavioral states similarly to both cold and hypertonic stressors, and psilocybin prevented such changes. These results indicate the crucial roles of HTR2 receptor pathways in behavioral changes following stress exposure and demonstrate that the observed stimulatory and anxiolytic effects of psilocybin likely occur from the modulation of such endogenous serotonergic pathways in the zebrafish brain.

The inventors have developed a high-resolution tracking system and a machine-learning framework for evaluating how a drug (e.g., psilocybin) changes the latent behavioral states of a model animal (e.g., larval zebrafish). Psilocybin has stimulatory effects on spontaneous swimming and preventative effects for stress-induced behavioral changes.

As shown in FIG. 4F, the inventors have demonstrated that these effects converged toward an intermediate state between spontaneous exploration and visually driven, rapid swimming in the latent behavioral space, indicating that psilocybin induces unique neural dynamics that are both stimulatory and anxiolytic. These observations have similarities with those in mammals, indicating the presence of common neural mechanisms in the evolutionarily conserved brain areas through which psilocybin exerts its behavioral effects.

The inventors have tracked precise body kinematics in a large environment, in order to produce the findings in this study. The 90 mm arena facilitated straight swim patterns compared to frequent turning/escape behaviors in a small arena, and allowed to identify distinct behavioral states that affect spontaneous exploration, visually driven rapid scooting and irregular swim patterns after stress exposure.

These results indicate that high throughput assays in small arenas such as multiwell plates may impair the full spectrum of the model animal's (e.g., zebrafish) behavioral repertoires. Moreover, as shown in FIGS. 1, 4 and 4S, the inventors have demonstrated that environmental stimuli that evoke different types of body kinematics, such as moving stimuli and acute stress exposure, could yield similar changes in macroscopic locomotion measures such as swim distance per minute. Precise tracking of body kinematics enabled robust inference of the shifts of latent behavioral states during stress exposure and psilocybin administration.

The inventors' observations open up new opportunities for further investigations into subcortical neural mechanisms by which psilocybin affects behaviors. While psilocybin is known primarily as an agonist for type 2 serotonin receptors, it also activates HTR1 receptors to exert its behavioral effects. Type 1 receptors are densely expressed in the brainstem areas of mammals and zebrafish, whereas type 2 receptors densely express in cerebellar areas of mammals and zebrafish. Therefore, psilocybin likely acts on these receptors to alter neural dynamics in the brain of zebrafish.

It is further important to investigate how does psilocybin stimulate swimming in a partially similar manner to visual stimuli. Neural mechanisms that trigger spontaneous swimming remain mostly elusive in zebrafish. Recent studies found that spontaneous activation of a sensory neural ensemble in the optic tectum triggers spontaneous swimming. Therefore, it is possible that psilocybin stimulates a part of the sensorimotor reflex circuit to induce swim patterns that are partially similar to those during visual stimulus motion (FIG. 3). It is also possible that persistent activation/suppression of motor circuits underlies such behavioral changes. Optogenetic activation of cerebellar Purkinje neurons induced swimming in zebrafish and locomotion in mice. Further investigation into neural dynamics based on whole-brain neural activity imaging methods and histological neural activity mapping methods will be necessary to disambiguate these potential mechanisms.

Psilocybin's preventative effects for stress-induced behavioral changes may occur from the same neural mechanisms responsible for its effect on spontaneous explorations, as the inventors have found that both seem to bring the fish's swim patterns toward an intermediate state between spontaneous exploration and visually driven rapid scooting (refer to FIG. 4F). Such common mechanisms can occur at neural circuit levels and molecular levels. Acute administration of psilocybin increases cortisol levels in humans despite its anxiolytic effects, indicating that psilocybin may not act directly on the hypothalamic-pituitary-adrenal axis and rather makes neural dynamics in the brain resilient to stress exposure. Such effects may occur through the cerebellum system. Patients with cerebellar neurodegeneration suffer from depressive symptoms, and the therapeutic effects of cerebellar stimulation have been clinically demonstrated. Chronic administration of serotonin-reuptake inhibitors increased functional connectivity between the cerebellum and midbrain structures in depression patients. These insights point to the pivotal roles of serotonergic modulation of the cerebellum in mood-related disorders. To address these questions, further investigations into brain-wide neural dynamics during acute stress exposure and psilocybin treatment may be done by implementing these behavioral paradigms into whole-brain imaging setups for head-fixed zebrafish or freely swimming zebrafish.

Psilocybin and other HTR2 agonists are effective in reversing depression-like behaviors after chronic stress exposure in rodents and humans, and a few doses have lasting effects. The latter persistent effect is unique to psilocybin compared to other antidepressants such as ketamine and SSRIs, but its underlying mechanisms are largely unknown. The inventors' findings pave the way to examine serotonergic psychedelics' unique pharmacological actions in the brains of larval zebrafish, which allow for live tracking of neural activity, neurotransmitters, structural plasticity, and molecular dynamics across the brain.

The inventors have used a custom-built zebrafish tracking system consisting of a high-speed camera, a macro lens and associated locking sleeve, an infrared filter for the lens, 880 nm LED illumination, a 100 mm×120 mm cold mirror, and a compact projector.

The inventors have tested the behavior of AB fish at the age of 5 Days Post Fertilization (DPF) on a chemical watch glass (125 mm) whose bottom surface was manually coated with a white spray. The inventors have imaged an area that spanned 90 mm (corresponding to 1100-1200 pixels) for each dimension with a resolution of 83 m per pixel at 290 Hz. The inventors recorded the behavior of each fish (one fish per experiment) for 15 minutes, which resulted in >250,000 frames. Behavioral data presented in this study were acquired using at least three batches, as depicted in FIGS. 1, 2, 3, 4, 1SD, 2H, 2I, 2J, 3L, 4S, for at least two batches (FIG. 3J).

Until 5 DPF, both control and drug-exposed embryos were reared in 90-mm Petri dishes and maintained in a light-cycled incubator at 28.0° C. Media was changed every other day and no food was given before behavioral experiments. All behavioral experiments in this study were performed in E3 medium after washing out pretreated drugs.

Psilocybin solution (Sigma, P-097) was purchased as a stock solution concentration of 1.0 mg/mL in acetonitrile:water (1:1), ampule of 1 mL (3.52 mM). Stock solutions were stored at −80° C. for long-term storage, and in-use aliquots were stored at −20° C. Aliquots were thawed and vortexed immediately before use. Psilocybin was administered by incubating the fish in the psilocybin solution in 6-well plates. Desired concentrations of psilocybin were achieved by adding the stock solution to the E3 medium in which the fish swim. Concentrations ranging from 1 μM to 50 μM were tested in order to optimize the dosage.

The inventors determined that a dose of 2.5 μM for 4 h had the largest effect on spontaneous swimming compared to controls (refer to FIGS. 3E and 3J). Following the 4-hour exposure, fish were double washed in E3 medium, and remained in E3 medium until their behavior was examined. Behaviors were recorded for 15 minutes per fish.

Fluoxetine hydrochloride (Sigma, F918) was prepared as a 1 mg/ml (2.9 mM) stock solution in a conditioned E3 medium for zebrafish embryos. Fluvoxamine (Sigma, F2802) was prepared as a 10 mg/ml (23 mM) stock solution in a conditioned E3 medium. Desired Fluoxetine and Fluvoxamine concentrations were achieved by diluting the stock solution with conditioned E3 medium and storing the aliquots at −20° C. At 5 DPF, the fish were incubated as follows: Fluoxetine and Fluvoxamine were administered by incubating the fish in the solutions in 6-well plates. Desired concentrations of the two types of SSRIs were achieved by adding the stock solutions to the E3 medium in which the fish swim. Concentrations ranging from 1 μM to 10 μM for Fluoxetine and 2.5 μM to 25 μM for Fluvoxamine were tested in order to optimize the dosage. After 24 hours of incubation the fish, after being triple washed, were transferred to a new Petri dish, similar to the one they dwelled in before, containing only E3 medium. Behaviors were recorded at 6 DPF for 15 minutes per fish.

The inventors utilized two established larval zebrafish stress paradigms to induce stress in the fish; hyperosmotic stress and cold temperature stress.

Zebrafish are freshwater fish, and thus a salt-water environment induces psychological and physiological stress. In order to create an osmotic environment, NaCl was dissolved in E3 medium in concentrations of 25 mM, 50 mM, and 100 mM NaCl, which have previously been found to induce stress in zebrafish (FIG. 4I). Fish were placed in the osmotic solution for 15 minutes and were then triple washed immediately before behavioral recordings.

Just as osmotic changes to the water cause stress to the fish, changes in water temperature are also known to induce stress. Zebrafish's optimal environment is approximately 28° C., and so it has been shown that a short exposure of 5 minutes to 18° C. leads to increased cortisol levels and anxiety-like behaviors in larval zebrafish. Thus, the inventors utilized this established stress paradigm and exposed the fish to 18° C. E3 medium for 5 minutes. The inventors subsequently returned the fish to 28° C. E3 for a 5-minute recovery before testing (FIG. 4A). 18° C. E3 was achieved by mixing 4° C. E3 with 28° C. E3. Over the course of the 5-minute cold stimulus, the water temperature on average ranged from 18° C. to 20° C. The recovery in 28° C. E3 was done in an incubator, thus the temperature remained stable. Note that these procedures involve multiple occasions of transferring fish and replacing liquid around the fish, which themselves cause stress response due to mechanical disturbances. The effect of such procedural stress was present as the shifts of behavioral states along the IC2 axis in the data presented in FIGS. 4 and S4 compared to other datasets.

For Ketanserin experiments (FIG. 4M), the inventors performed a bath application of Ketanserin (11.25 μM) for 5 hours before behavioral tests. Psilocybin (2.5 μM) was added to the solution 1 hour after the start of Ketanserin treatment for a total duration of 4 hours before behavioral tests. Ketanserin (Sigma, S006) was prepared as a 0.5 mg/mL stock solution in E3 medium. Stock solutions were stored at −80° C. for long-term storage, and in-use aliquots were stored at −20° C. The desired concentration of Ketanserin was achieved by adding the stock solution to the E3 medium in which the fish swim.

The inventors have used custom Python scripts to extract swimming parameters and tail movements. The following procedures were applied to each data:

In a first step, the inventors identified the pixel-level centroid position (denoted herein as centroid 127C) of the head of the fish for each frame. The inventors proceeded to extract square patches or segments (denoted herein as segments 127S) around the fish. To do this, the inventors calculated the average background image based on 100 images equidistantly sampled from all time points and subtracted it from the movie. The inventors applied a gaussian blur filter (σ=250 m) to background-subtracted images and identified the darkest pixel as the centroid position of the head of the fish. The inventors cropped the image (144 by 144 pixels, 12×12 mm) around the centroid, rescaled it to between 0-255 brightness values, and stored them in a separate AVI file. To accelerate file processing time, we used a recurrent algorithm that searched proximities of the fish positions of previous time points.

In a second step, the inventors used a deep neural network, (also referred to herein as ML model 125), automatically identify, or annotate (denoted herein as 125AN) body parts from the extracted fish images. The inventors trained the network by using 550 manually annotated images (FIG. 2E). During the development process, the inventors observed that biases in the training datasets were reflected in the automatic annotation results. Therefore, the inventors balanced the repertoires of training images so that they covered all angles of the tail symmetrically between the left and right sides. The inventors also included images with outlier pixels, which result from a small inhomogeneity of the bottom coating of the dish. Errors in manual annotations were further screened and corrected by independent algorithms that detect significant deviations in distances between annotated body parts.

In a third step, the inventors applied [i] sub-pixel head centroid detection and [ii] tail angle quantification for each frame. For sub-pixel head centroid detection (FIG. S1C), the inventors applied a gaussian blur filter to the fish image and applied a sub-pixel centroid detection algorithm. This identification of sub-pixel level centroid position facilitated visualizing of swim trajectories, and also for extracting tail motion parameters during swim bouts.

For tail angle quantification (FIG. 2F), the inventors used a quadrature module (denoted herein quadrature module 120), to fit a quadratic function to seven annotated points 125AN along the body trunk and the tail, and quantified the angle of the fit function relative to the body-nostril axis. The angle quantification was made at 1 mm from the base part of the tail. the inventors experimentally found that quadratic fit provides optimal signal-to-noise ratio in this low-resolution imaging system.

In a subsequent step, the inventors identified each swim bout based on swim velocities and quantified basic motion features 130MF, such as position, velocity, duration, moved distance, and tail kinematic parameters (frequency, number of tail motions, angles of tail motions), etc.

The inventors have found that lateral motions of sub-pixel head centroid relative to the direction of the swimming, which synchronously precedes tail motions, provide a slightly better signal-to-noise ratio for defining individual cycles of tail motions in this low-resolution imaging system. Therefore, the algorithm used the motion of the sub-pixel head centroid as a reference to read out maximum tail angles for each tail motion cycle (FIG. 2G). Motion features 130MF such as peaks of tail angles may be detected for each tail motion cycle, and may further be averaged across cycles to quantify average tail angles. Motion features 130MF such as tail frequencies may be quantified based on these tail motion cycles. Motion features 130MF such as left/right (L/R) balance of the tail motion may be calculated according to equation Eq. 1, below:

$$L / R Balance = \vert \frac{Σ ( Right tail angles ) - Σ ( Left tail angles )}{Σ ( Right tail angles ) + Σ ( Left tail angles )} \vert$$

such that the value becomes 0 if the tail motions are symmetric, and 1 if there is only one tail movement to a specific side.

The inventors have tested the accuracy of tail motion tracking by examining how accurately swim distances may be predicted based on tail motion parameters (FIG. 2H). The inventors created a multiplicative model that predicts the distance of swimming based on the frequency, number, and average angle of tail motions for each swim bout. The inventors extracted forward swim events that occurred in the central part of the large dish (30 mm from the center) and caused changes in the head direction less than 30 degrees. The inventors fit the model by using the L-BFGS-B method implemented in the minimize function of Scipy package and identified optimal values for the power factors for each tail parameter across 20 fish. The optimal power factors were roughly 0.4 for the tail angle and 1 for the frequency and the number of tail motions. The inventors subsequently calculated the correlation coefficient with the actual swim distance and the predicted distance from tail motions (FIG. 2H).

The inventors used custom Python scripts to summarize the results presented in this study. Except for the analysis of swimming distances (FIGS. 1D, 3E, 4B, 3J, 3L and 4I), the inventors only included swim episodes that occurred in the central part of behavioral arenas (within 10 mm or 30 mm from the center of the small or large arena, respectively) for the analyses of swim/tail parameters throughout this study to rule out the physical effect of the wall in the small arena and shallow places in the large arena.

Statistical tests for swimming distances, head angle changes and individual tail parameters (FIGS. 3F, 4E, 2J, 2H) used either 2-sample t-test or Tukey's post-hoc test followed by one-way analysis of variance (ANOVA) in the Scipy package (https://scipy.org/).

For Independent Component Analysis (ICA) (FIGS. 2D, 3G, 4D, 4K and 4L), The inventors identified ICA weights and normalization factors in the dataset of 14,694 swimming episodes from N=22 fish for the small arena and N=20 fish for the large arena (FIG. 1) by using FastICA function in scikit-learn package (https://scikit-learn.org). The inventors subsequently applied the same ICA weights and normalization factors to other datasets. Scatter density plots in this study (FIGS. 2D, 3G, 4D, 4K, 4L and 4M) were generated using gaussian_kde function in Scipy package for dot coloring and kdeplot function in Seaborn package (https://seaborn.pydata.org/) for contour lines. Statistical tests for distributional differences of independent components (FIGS. 3H, 4D, 2I, 4K, 4L, 4M) were performed using kernel density 2-sample test (kde.test) in R package through rpy2 Python-R bridge (https://pypi.org/project/rpy2/). The inventors used this density-based test instead of Kolmogorov-Smirnov test because the former provides more conservative levels of significance for larger sample sizes.

The sequence homology analysis of serotonin receptors between zebrafish and human (FIGS. 3B and S3A) was performed using Clustal Omega algorithm and iTOL visualization tool on the EMBL website. Uniplot IDs used for this analysis are as follows. Human serotonin receptors: hHTR1A (P08908), hHTR1B (P28222), hHTR1D (P28221), hHTR1E (P28566), hHTR1F (P30939), hHTR2A (P28223), hHTR2B (P41595), hHTR2C (P28335), hHTR3A (P46098), hHTR3B (095264), hHTR3C (Q8WXA8), hHTR3D (Q70Z44), hHTR3E (A5X5Y0), hHTR4 (Q13639), hHTR5A (P47898), hHTR6 (P50406), hHTR7 (P34969). Zebrafish serotonin receptors: zHTR1aa (A0A8M1NIJ6), zHTR1ab (A0A8M1NRS3), zHTR1b (B3DK14), zHTRld (A0A8M2B5P5), zHTR1e (A0A8M9P2V8), zHTR1fa (A0A8M6Z176), zHTR1fb (A0A8M2B6K6), zHTR2aa (A0A8N7TD42), zHTR2ab (A0A8M3B093), zHTR2b (Q0GH74), zHTR2cl1 (A0A8M6Z717), zHTR2cl2 (A0A8M1PZA4), zHTR3a (A0A8M9PD95), zHTR3b (A0A8M9PJB8), zHTR4 (A0A8M9QPE9), zHTR5aa (A0A8M1NJ85), zHTR5ab (Q7ZZ32), zHTR6 (A0A8M3ANX4), zHTR7a (A0A8N7T7N6), zHTR7b (A0A8M9QGY4), zHTR7c (A0A8M1RQY0).

zHTR2cl1 expression map (FIG. 3C) was constructed using RNA fluorescence in situ hybridization (RNA-FISH) with hybridization chain reaction (HCR) method 109. HCR probe for zHTR2cl1, amplifiers and buffers were purchased from Molecular Instruments. The staining was performed according to an HCR protocol by using a B3 amplifier with Alexa Fluor 546. The inventors used 5-day old transgenic zebrafish that pan-neuronally express nuclear-localized, genetically encoded calcium indicator (Tg(HuC:H2B-GCaMP7f)) for the staining to register the volumetric image to a reference brain based on GCaMP expression.

Labeled fish were imaged in a custom light-sheet microscope. Prior to the imaging, fish were embedded in 2% low-melting agarose in fish water on a custom-made pedestal inside a glass-walled chamber. Agarose around the head was removed with a microsurgical knife (#10318-14, Fine Science Tools) to minimize the scattering of the excitation laser. For each fish, we acquired two images: Tg(HuC:H2B-GCaMP7f) channel and the zHTR2cl1 HCR staining image.

The inventors created an average image for each fish, and then identified individual neurons that express nuclear-localized GCaMP based on the average image by using an algorithm for detecting circular shapes in images. The inventors registered the GCaMP image channel of each fish to Tg(elavl3:H2b-GCaMP6s) reference image from mapZebrain database using Advanced Normalization Tools (ANTs), and applied the same registration to the HCR in situ image. For each fish, the inventors analyzed the HCR signal in the coordinates of the recognized neurons after subtracting the local background and created a binarized image of the signal. The inventors subsequently overlaid a spherical gaussian filter of the positive neurons of each fish, normalized by the number of cells, to create a generalized 3D image of expressing regions.

The inventors investigated whether psilocybin affects neural dynamics in the brain of larval zebrafish by examining the neural population dynamics in the dorsal raphe nucleus (DRN). Acute administration of lysergic acid diethylamide (LSD, HTR2 agonist) and psilocin both induced rapid suppression of serotonergic neurons in the DRN in mammals. It was investigated whether psilocybin induces similar changes in zebrafish by using a head-fixed virtual reality setup and calcium imaging of neural activity (FIG. 6A). In this setup, an immobilized zebrafish larva is placed in an imaging chamber. We record swim signals from spinal motoneurons by using a pair of electrodes attached to the tail while we project moving visual stimuli beneath the fish. Both control and psilocybin-treated fish stopped swimming or showed only occasional spontaneous swimming when the visual stimuli stopped (“Spontaneous” period), and both showed vigorous swimming (FIG. 6B) when the visual stimuli moved forward (“OMR” period). Neural activity was recorded in the DRN by using a bespoke light-sheet microscope during these two task periods using transgenic zebrafish that express nuclear-localized calcium indicators pan-neuronally (FIG. 6C).

The dorsal raphe nucleus in zebrafish mainly consists of two neural populations: serotonergic neurons that mainly reside along the midline and GABAergic neurons that mainly reside in the lateral part (FIG. 6D). In a previous study the inventors demonstrated that serotonergic and GABAergic neurons in the DRN have complementary activity patterns, where the former population respond to pause of swimming or backward optic flow during swimming, and the latter population encodes the strength of swimming. Consistently, it was found that neurons along the midline (predominantly serotonergic neurons) showed higher activity during the spontaneous period when the fish mostly stopped swimming. Neurons in the lateral part of the DRN (predominantly GABAergic neurons) showed higher activity during the OMR period when the fish showed vigorous swimming (FIG. 6E).

It was further investigated how psilocybin treatment changes such neural population dynamics in the DRN. The fraction of neurons that showed higher activity during the spontaneous period (Class 1 neurons), which are predominantly serotonergic, became significantly lower after psilocybin treatments in the DRN (FIG. 6F). On the contrary, the fraction of neurons that showed higher activity during the OMR period (Class 2 neurons), which are predominantly GABAergic, became slightly higher. These results suggest that acute psilocybin exposure inhibits serotonergic neurons in the zebrafish DRN, potentially by activating nearby GABAergic neurons.

To visualize the shifts in such complementary dynamics between different neural populations, non-negative matrix factorization (NMF) was applied to the trial-averaged activity of all neurons in the DRN and plotted the transition of neural states based on the first two identified components (FIG. 6G). The non-negative constraint of this dimensionality reduction method is suited to separate complementary activity patterns of two neural populations. The inventors pooled trial-averaged activity patterns of all neurons in the dorsal part of the DRN from all tested fish and fit NMF (components=2) to obtain two weights of each neuron for two non-negative population vectors. The vectorial inner products of neuronal weights and their mean ΔF/F values was then calculated for each time point in the task within each fish (thin lines) or from each group of fish (thick lines) to show neural state transitions between two components. In the control fish, neural states in the DRN shift rightward along the horizontal axis (NMF #2) during the spontaneous period by the activation of Class 1 neurons. Fish's vigorous swimming during the OMR period shifts up the neural state along the vertical axis (NMF #1). Psilocybin treatment diminished the shift of the neural state along the horizontal axis and instead enhanced its shift along the vertical axis. These results show that psilocybin suppresses neural activations in the DRN during the pause of swimming, which indicates the suppression of serotonergic neurons (FIG. 6G).

The resemblance of this phenomenon with mammalian observations suggests that psilocybin triggers similar changes in neural dynamics in brain structures evolutionarily conserved between teleosts and mammals.

Reference is now made to FIG. 7, which is a block diagram depicting a computing device, which may be included within an embodiment of a system for determining efficacy of a treatment, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task, or script. Executable code 5 may be executed by processor or controller 2 possibly under control of operating system 3. For example, executable code 5 may be an application that may determine efficacy of a treatment as further described herein. Although, for the sake of clarity, a single item of executable code 5 is shown in FIG. 7, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data pertaining to depiction of an animal may be stored in storage system 6, and may be loaded from storage system 6 into memory 4 where it may be processed by processor or controller 2. In some embodiments, some of the components shown in FIG. 7 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components, or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

Reference is now made to FIG. 8, which depicts a system 10 for determining efficacy of a treatment, according to some embodiments of the invention.

According to some embodiments of the invention, system 10 may be implemented as a software module, a hardware module, or any combination thereof. For example, system 10 may be or may include a computing device such as element 1 of FIG. 7, and may be adapted to execute one or more modules of executable code (e.g., element 5 of FIG. 7) to determine efficacy of a treatment, as further described herein.

As shown in FIG. 8, arrows may represent flow of one or more data elements to and from system 10 and/or among modules or elements of system 10. Some arrows have been omitted in FIG. 8 for the purpose of clarity.

As shown in FIG. 8, system 10 may include, or may be communicatively connected (e.g., via the Internet) to at least one imaging device 20 (also referred to herein as camera 20). Imaging device 20 of FIG. 8 may be the same as camera 20 of FIG. 1A, and may be, or may include a high-speed Infrared (IR) camera or visible light camera, as elaborated herein.

According to some embodiments, system 10 may receive from the at least one imaging device or camera 20, one or more (e.g., a plurality of) images 25 depicting motion of an animal 25′. As elaborated herein (e.g., in relation to FIG. 1A), animal 25′ may be a model animal of interest, such as a zebrafish, that may be treated with a predetermined substance of interest, such as Psilocybin.

As shown in FIG. 8, system 10 may include a detection module 110, configured to detect at least one object depicted in image 25. For example, as depicted in FIG. 1A, detection module 110 may be configured to produce a segment 110S of image 25, which includes a depiction of animal 25′.

As shown in FIG. 8, system 10 may include a motion feature extraction module 130, configured to extract, from images 25, one or more (e.g., a plurality of) motion features 130MF, representing motion of at least one specific body part (herein denoted 130MF1) of animal 25′ and/or motion of animal 25′ (herein denoted 130MF2).

Pertaining to the example where animal 25′ is a fish (e.g., a zebrafish), motion features 130MF may include, for example tail motion features 130MF1, such as (i) a frequency of tail motions, (ii) an amplitude of tail motions, (iii) an angle of tail motions, (iv) a number of tail motions in a predetermined timeframe, (v) a balance of tail motions between a left side and a right side of the fish, and the like.

Additionally, or alternatively, and pertaining to the example where animal 25′ is a fish (e.g., a zebrafish), the plurality of motion features 130MF may include, for example head motion features 130MF1 such as (vi) a frequency of head motions, (vii) an angle of head motions, and (viii) a number of head motions within a predefined timeframe.

Additionally, or alternatively, and pertaining to the example where animal 25′ is a fish (e.g., a zebrafish), the plurality of motion features 130MF may include, for example fin motion features 130MF1 and/or eye motion features 130MF1, including for example (ix) a frequency of fin motions, (x) an amplitude of fin motions, (xi) an angle of eye motions, and (xii) a frequency of eye motions.

Additionally, or alternatively, and pertaining to the example where animal 25′ is a fish (e.g., a zebrafish), the plurality of motion features 130MF may include motion features 130MF2 that pertain to motion, or traversal of animal 25′ within arena 20AR, also referred to herein as swim interval features 130MF2. Swim interval features 130MF2 may include for example a duration of a swim episode, and an interval between swim episodes.

As elaborated herein, e.g., in relation to the experimental setups of FIGS. 1A-1C, the constraint of monitoring a large arena 20AR (e.g., to diminish space-related stress) may result in low pixel resolution, which may impede accuracy of motion feature 130MF extraction. Therefore, system 10 may be configured to employ a data processing algorithm that facilitates tracking of head trajectories and tail kinematics at sub-pixel resolution, e.g., at spatial scales smaller than the size of pixels depicting animal 25′, as elaborated herein.

As shown in FIG. 8, system 110 may include a sub-pixel centroid module 127 (or centroid module 127, for short), an ML-based sub-pixel model 125 (or “sub-pixel model 125”, for short), and a quadrature module 120. As elaborated herein, modules 129, 125 and 127 may be configured to collaborate with motion feature extraction module 130, to produce at least some of motion features 130MS, as elaborated herein.

According to some embodiments, centroid module 127 may be configured to identifying one or more body parts 127C of the depicted fish 25′ in images 20. For example, centroid module 127 may identify a pixel-level centroid position 127C of a head of fish 25′ for one or more (e.g., each) image 20. Additionally, or alternatively, centroid module 127 may extract a segment 127S, or a square patch around animal 25′ (e.g., boxing the depicted fish 25′), as elaborated herein.

According to some embodiments, ML model 125 may be configured to automatically determine, or annotate locations 125AN of specific points of the fish, at sub-pixel resolution, based on segments 127S and/or the one or more body parts 127C.

As elaborated herein (e.g., in relation to FIG. 2E), ML model 125 may be pretrained by a supervised training process, using manually annotated images or segments 127S, that include a pixel-level centroid position 127C of a head of a depicted fish 25′. In a subsequent reference stage, system 10 may apply ML model 125 to determine or annotate these locations of specific points on a target image depicting a fish, at sub-pixel resolution, as shown in FIG. 1A.

For example, system 10 may receive (e.g., via input device 7, and/or via database 6 of FIG. 8) a training dataset that may include a plurality of images 25 or segments 127S depicting a fish 25′. System 10 may present, via a user interface such as output device 8 of FIG. 7, images 25 or segments 127S, and prompt a user to provide a relevant annotation 125ANM, describing a pixel-level centroid position 127C of a head of the depicted fish 25′. System 10 may subsequently use the plurality of manual annotations 125ANM as supervisory data, to train sub-pixel ML model 125, to determine or annotate these locations of specific points on a target image depicting a fish, at sub-pixel resolution, as shown in FIG. 1A.

Additionally, or alternatively, system 10 may train ML model 125 using a semi-supervised process. As explained herein, images 25 or segments 127S may depict fish 25′ in a sub-pixel resolution, e.g., where features and body parts of the animal are defined by spatial scales that are smaller than the size of pixels.

According to some embodiments, system 10 may include an ML-based high-resolution feature extraction model 170. System 10 may receive a training dataset that includes high-resolution images 25HR. ML feature extraction model 170 may be pretrained to automatically identify features such as body parts of depicted fish 25′ in high resolution images 25HR. System 10 may infer, or apply ML feature extraction model 170 on the high-resolution images 25HR, to automatically obtain annotations 125ANA defining features or body parts of depicted fish 25′ in high resolution images 25HR. System 10 may down sample the images to a resolution befitting sub-pixel ML model 125, and may use automatically obtain annotations 125ANA as supervisory information, to train ML model 125 in a semi-supervised process.

According to some embodiments, quadrature module 120 may be configured to fit the determined locations (e.g., points on the depicted fish 25′) into a quadratic curve, as shown in the example of FIG. 2F.

Quadrature module 120 may quantify motion of the at least one body part based on the quadrature fitting, and may calculate a value of at least one motion feature 130MF based on this quantification. In the example of FIG. 2F, for tail angle quantification (denoted θ), quadrature module 120 may fit the quadratic function to seven annotated points 125AN along the body trunk and the tail, and quantify the angle (θ) of the fit function relative to the body-nostril axis. In this example, the value of tail angle θ may be the same as that of tail angle motion feature 130MF.

Module 130 may use the quantification of tail angle θ to quantify other motion feature values which represent motion body parts. For example, module 130 may compute a motion feature 130MF representing a rate of tail strokes, based on location of one or more specific points 125AN of the fish on the quadratic curve.

In another example, module 130 may compute a motion feature 130MF representing an amplitude of tail strokes, based on location of one or more specific points of 125AN the fish on the quadratic curve.

In another example, module 130 may compute a motion feature 130MF representing a rate of head movements, based on location of one or more specific points 125AN and/or 127C of the fish, in relation to the quadratic curve.

In another example, module 130 may fine-tune, or compute the rate of tail strokes 130MF further based on the identified sub-pixel centroid location 127C of the head of the depicted fish. In other words, head motion 130MF and tail motions 130MF may be quantified separately with independent algorithms, and these two pieces of information may later be combined to calculate a robust, low noise behavioral indicator. This combination of tail motion and head motion information is based on the observation that head movements and tail movements are highly synchronized, as shown in the example of FIG. 2G.

According to some embodiments, system 10 may include a dimensionality reduction module 140, configured to apply a dimensionality reduction algorithm, such as Independent Component Analysis (ICA) algorithm on the plurality of motion features 130MF, to obtain a latent vector 140LV, that includes a plurality of latent features 140LF. It may be appreciated that latent vector 140LV may be, or may include a representation of the plurality of motion features 130MF in a latent space 140LS.

It may be appreciated by a person skilled in the art that transfer of motion features 130MF into latent vectors 140LV in latent space 140LS, and subsequent analysis of these latent vectors 140LV, instead of focusing on noisy individual motion features 130MF, may facilitate robust estimation of behavioral aspects of the examined animal 25′.

For example, dimensionality reduction module 140 may be, or may include an autoencoder model 140. As known in the art, an autoencoder model (e.g., 140) may include a first portion, commonly referred to as an encoding portion, or encoder 140ENC, and a second portion, commonly referred to as a generative portion, or decoder 140DEC. Encoder 140ENC may be trained to encode incident sample data, such as motion features 130MF into a latent feature vector 140LV representation, in a reduced-dimension latent space 140LS. Latent vector 140LV may include a plurality of entries, referred to herein as latent motion features 140LF, or latent features 140LF for short.

The generative portion (e.g., decoder) 140DEC may be trained, e.g., in parallel to, or intermittently with encoder 140ENC, to decode the latent feature vectors 140LV, so as to produce a reconstructed version of the incident motion features 130MF data, via latent vector 140LV. Latent vector 140LV may be characterized by a reduced dimension (e.g., a number of member latent motion features 140LF), in relation to a dimension of the incident sampled data (e.g., the number motion features 130MF). Therefore, the reconstructed version of the input motion features 130MF may be regarded as filtered by the latent space of dimensionality reduction module 140, as defined by the plurality of latent features 140LF.

Additionally, or alternatively, dimensionality reduction module 140 may include, or may be implemented by another appropriate algorithm of dimensionality reduction as known in the art, such as an Independent Component Analysis (ICA) algorithm, to obtain latent motion vector 140LV as an embedding, or a compressed representation of input motion features 130MF. The terms “ICA”, or “ICA component”, and “latent feature 140LF” may be used interchangeably in this context.

As elaborated herein, system 10 may calculate a value of a behavioral indicator 160BI, representing a behavior of animal 25′, based on the latent features 140LF of the latent feature vector 140LV. System 10 may subsequently determine efficacy of treatment of animal 25′ (e.g., zebrafish) by the substance of interest (e.g., Psilocybin), based on the behavioral indicator 160BI value.

For example, and as shown in FIG. 8, system 10 may include a machine learning (ML) based model 150, also referred to herein as classifier 150. Classifier 150 may be pretrained to classify latent vector 140LV to one or more classes of movement patterns 150MP. In other words, ML model 150 may be trained to map latent vectors 140LV (and, inherently, member latent features (e.g., ICAs) 140LF) into classes 150MP that represent movement patterns 150MP. Such mapping is demonstrated herein, e.g., in relation to FIGS. 2D and 2I, where combinations of latent features 140LF (denoted ICA1 and ICA2) are mapped to motion patterns 150MP such as a scooting motion pattern 150MP and a turn/escape motion pattern 150MP.

In the non-limiting example where the model animal 25′ is a fish such as a zebrafish, classes of movement patterns 150MP may include, for example short scooting, rapid long scooting, performance of routine turns, performance of C-turns, patterns of immobility, patterns of intermittent mobility, and the like.

Additionally, or alternatively, motion patterns 150MP may include organ movement patterns 150MP. In the non-limiting example where the model animal 25′ is a fish, such organ movement patterns 150MP may include an eye movement pattern (e.g., frequency and direction of eye movement), a heart movement pattern (e.g., mean and variance of heart rate), a fin movement pattern (e.g., direction, mean and variance of fin movement), and limb movement pattern (e.g., direction, mean and variance of limb movement).

According to some embodiments, classifier ML model 150 may be trained through a supervised training process, as known in the art.

For example, system 10 may receive (e.g., via input device 7, and/or via database 6 of FIG. 8) a training dataset 150DS. Dataset 150DS may include a plurality of time-based, or time-stamped sequences 140LVS of one or more latent vectors 140LV.

Additionally, system 10 may obtain a plurality of movement pattern annotations 150DS′, corresponding to the plurality of latent vector sequences in dataset 150DS. For example, system 10 may present, via a user interface such as output device 8 of FIG. 7, a sequence of images 25 (e.g. a movie), and prompt a user to provide a relevant annotation 150DS′, describing a movement pattern 150MP (e.g., scooting, turning, etc., as elaborated herein) performed by animal 25′ in that sequence.

System 10 may subsequently use the plurality of movement pattern annotations 150DS' as supervisory data, to train ML model 150, to classify latent vectors 140LV of at least one sequence 140LVS to one or more classes 150MP of movement patterns.

System 10 may perform this training during a training period, subsequently replaced by an inference period, where classifier 150 may be applied to samples of target images 25, to ascertain their movement pattern classification 150MP. Additionally, or alternatively, system 10 may continuously (e.g., repeatedly over time) train classifier 150, as additional, new images 25 are introduced into system 10.

Additionally, or alternatively, system 10 may include a behavioral model 160. Behavioral model 160 may be configured to calculate a behavioral indicator 160BI value based on the classification of movement patterns 150MP.

Classifier 150 may classify a latent vector 140LV of an animal 25′ of interest to one or more classes of movement patterns 150MP, and associate a confidence level, or confidence score 150MP′ to each classification 150MP. Behavioral model 160 may, in turn, calculate behavioral indicator 160BI value based on classifications 150MP.

For example, behavioral model 160 may calculate behavioral indicator 160BI as a weighted function of classifications 150MP, e.g., weighted by confidence scores 150MP′.

In another example, behavioral model 160 may be, or may include an ML model, such as a nonlinear, NN based model. Behavioral model 160 may receive (e.g., via input device 7 of FIG. 8) annotations of the animal's 25′ behavior, and may be pretrained to classify or predict the behavioral indicator 160BI, as representing a behavior of depicted animal 25′, based on classifications 150MP, using the annotations of the animal's 25′ behavior as supervisory information.

Behavioral indicator 160BI of an animal 25′ of interest may be, or may include one or more numerical scores representing levels of corresponding behavioral states. These behavioral states may include, for example anxiety of animal 25′, arousal of animal 25′ (e.g., when chasing food), sleepiness of animal 25′, responsiveness of animal 25′ to a visual stimulus, responsiveness of animal 25′ to an odor stimulus, responsiveness of animal 25′ to an acoustic stimulus, a motoric disability of animal 25′, an indication of appetite of animal 25′, and the like.

Additionally, or alternatively, behavioral model 160 may accumulate (e.g., in database 6 of FIG. 7) behavioral indicators 160BI obtained from a plurality of animals 25′ and/or the same animal(s) 25′ over time. For example, behavioral model 160 may accumulate (i) behavioral indicators 160BI of animals 25′ treated with a substance (e.g., drug) of interest (herein “treated” animals 25′), and (ii) behavioral indicators 160BI of animals 25′ that were not treated with the substance of interest (herein “control” animals 25′).

Behavioral model 160 may analyze, or compare accumulated behavioral indicators 160BI of treated animal 25′ vis-à-vis accumulated behavioral indicators 160BI of control animals 25′, to ascertain effect of the treatment of interest on the model animals 25′. In other words, behavioral model 160 may determine efficacy of treatment of the substance of interest on the model animal 25′, based on the behavioral indicator values 160BI.

For example, behavioral model 160 may compare a pre-treatment value of the behavioral indicator 160BI, with a post-treatment value of the behavioral indicator; and calculate a statistical significance value, representing correlation between application of the predetermined substance and the behavioral indicator, based on this comparison, to determine efficacy of the treatment.

Additionally, or alternatively, behavioral model 160 may produce a treatment efficacy data element 160TE. Treatment efficacy data element 160TE may include one or more numerical values, corresponding to respective behavioral states (e.g., anxiety of animal 25′, arousal of animal 25′, etc. as elaborated herein), each representing an effect of the substance and/or dosage of the substance of interest in changing the respective behavioral states.

Reference is now made to FIG. 9A which is a flow diagram, depicting a method of determining efficacy of treatment by at least one processor (e.g., processor 2 of FIG. 7) according to some embodiments of the invention.

As shown in step S1005, at a preliminary stage, a model animal (e.g., a zebra fish) 25′ may be treated with a predetermined substance or drug of interest, as elaborated herein.

As shown in step S1010, the at least one processor may receive, from at least one camera or imaging device (e.g., imaging device 20 of FIG. 8), images 25 depicting motion of the model animal 25′ treated with the predetermined substance.

As shown in step S1015, the at least one processor 2 may extract, from images 25, a plurality of motion features (e.g., 130MF, such as 130MF1, 130MF2 of FIG. 8). As explained herein, motion features 130MF may represent or define a plurality of quantified motion characteristics of at least one specific body part of the depicted animal 25′.

As shown in step S1020, the at least one processor 2 may apply a dimensionality reduction algorithm (e.g., 140 of FIG. 8) such as an autoencoder algorithm, or an ICA algorithm on the plurality of motion features 130MF, to obtain a latent vector 140LV. Latent vector 140LV may be comprised of a plurality of latent features 140LF, and may represent the plurality of motion features 130MF in a latent space.

As shown in step S1025, the at least one processor may calculate a value of a behavioral indicator 160BI, representing a behavior of animal 25′, based on the latent features 140LF of the latent vector 140LV.

For example, and as elaborated herein, the at least one processor 2 may employ an ML model (e.g., model 150 of FIG. 8), that may be pretrained to map latent vectors 140LV (and, inherently, member latent features (e.g., ICAs) 140LF) into classes, e.g., classes 150MP of FIG. 8. Classes 150MP may represent movement patterns 150MP such as traversal patterns (e.g., short scooting, rapid long scooting, performance of routine turns, performance of C-turns, a pattern of immobility, a pattern of intermittent mobility, and the like), and organ movement patterns (e.g., an eye movement pattern, a heart movement pattern, a fin movement pattern, a limb movement pattern, and the like). The at least one processor may apply pretrained ML model 150 to classify latent vector 140LV to one or more classes of movement patterns, and calculate the behavioral indicator value 160BI based on (e.g., as a weighted sum, or weighted function of) classification 150MP.

As shown in step S1030, the at least one processor 2 may determine efficacy of the treatment based on the behavioral indicator value. For example, the at least one processor 2 may calculate, and produce a treatment efficacy data element 160TE, that may include one or more numerical values. The numerical values of treatment efficacy data element 160TE may corresponding to respective behavioral indicator 160BI of behavioral states (e.g., anxiety of animal 25′, arousal of animal 25′, etc. as elaborated herein), and may represent an effect of the substance and/or dosage of the substance of interest in changing the respective behavioral indicators 160BI of behavioral states.

Reference is now made to FIG. 9B which is a flow diagram, depicting a method of screening for a compound suitable for treating a psychological state in a subject in need thereof, by at least one processor (e.g., processor 2 of FIG. 7) according to some embodiments of the invention.

As shown in step S2005, at a preliminary stage, a model animal 25′ may be administered with an effective amount of the compound of interest. For example, animal 25′ may be a fish (e.g., a zebra fish). In such embodiments administering may be performed via feeding, or via introduction of the compound of interest into a body of water in which the fish resides.

As shown in step S2010, and as explained herein, the at least one processor 2 may measure, or calculate motion features (e.g., 130MF, such as 130MF1, 130MF2 of FIG. 8). Motion features 130MF may represent or define characteristics of motion of at least one specific body part of the depicted animal 25′.

As shown in step S2015, and as explained herein, the at least one processor 2 may determine or calculate a latent vector 140LV, representing the plurality of motion features 130MF in a latent space. The latent vector 140LV may include a plurality of latent features, representing motion features 130MF in a latent space.

As shown in step S2020, and as explained herein, the at least one processor 2 may calculate a value of a behavioral indicator 160BI, representing a behavior of the administered animal 25′ based on the latent features 140LF of latent vector 140LV.

According to some embodiments, a behavioral indicator 160BI of the animal 25′ administered with the compound being equal to, or greater than a pre-determined threshold, may be indicative of the compound being suitable for treating the psychological state in a subject (e.g., a human subject) or patient in need thereof.

Additionally, or alternatively, a behavioral indicator 160BI of the animal 25′ administered with the compound being lower than a pre-determined threshold may be indicative of the compound being unsuitable for treatment.

Pertaining to the example of stress related medication, a first behavioral indicator 160BI value, representing a level of stress of administered animal 25′ may be compared to a corresponding second behavioral indicator 160BI value, in a control animal 25′ (e.g., one that has not been administered with stress medication). A difference between the first and second behavioral indicator 160BI values may indicate efficacy of the medication of interest, whereas a small difference, or an opposite difference (indicating increase in stress indicators 160BI) may indicate that the medication of interest is unsuitable for stress treatment.

As elaborated herein, embodiments of the invention may be utilized to automatically assess an efficacy of a substance of interest, or screen the substance of interest as either suitable or non-suitable for treatment of a predefined psychological condition (e.g., a state of stress). As such, embodiment of the invention may provide a practical application that may improve assessment of drugs in the technological fields of pharmaceutics and assistive diagnostics.

As can be seen, the present invention represents a method and system for determining efficacy of treatment, as well as a method of screening for a compound suitable for treating a psychological state in a subject in need thereof, which contribute to the improvement of the abovementioned technological field by providing highly reliable tool for evaluation of the behavioral effect in a subject (e.g., larval zebrafish) treated with a substance of interest, based on treatment-related imagery. The reliability of the suggested methods and systems has been confirmed by multiple tests, as demonstrated above.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1.-33. (canceled)

34. A method of determining efficacy of treatment by at least one processor, the method comprising:
receiving, from at least one camera, images depicting motion of an animal, wherein said animal is treated with a predetermined substance;
extracting, from said images, a plurality of motion features, each representing a specific quantified motion characteristic of at least one specific body part of the animal;
applying a dimensionality reduction algorithm on the plurality of motion features, to obtain a latent vector representing the plurality of motion features in a latent space, wherein the latent vector comprises a plurality of latent features;
calculating a value of a behavioral indicator, representing a behavior of the animal, based on the latent features of the latent vector; and
determining efficacy of the treatment based on the behavioral indicator value.

35. The method of claim 34, wherein the animal is a fish, and wherein said body parts are selected from a head of the fish, a tail of the fish, an eye of the fish and a heart of the fish.

36. The method of claim 34, wherein the plurality of motion features are selected from at least one of: (a) a list of tail motion features consisting of: a frequency of tail motions, an amplitude of tail motions, an angle of tail motions, a number of tail motions in a predetermined timeframe, a balance of tail motions between a left side and a right side of the fish; (b) a list of head motion features, said list consisting of: a frequency of head motions, an angle of head motions, and a number of head motions within a predefined timeframe; (c) a list consisting of a frequency of fin motions, an amplitude of fin motions, an angle of eye motions, and a frequency of eye motions; and (d) a list of swim interval features, said list consisting of: a duration of a swim episode, and an interval between swim episodes.

37. The method of claim 34, further comprising:
applying a pretrained, first machine learning (ML) based model, to classify the latent vector to one or more classes of movement patterns; and
calculating the behavioral indicator value based on said classification.

38. The method of claim 37, further comprising training the first ML based model, said training comprising:
receiving a training set, comprising a plurality of time-based sequences of one or more latent vectors;
obtaining a plurality of movement pattern annotations, corresponding to the plurality of latent vector sequences; and
using the plurality of movement pattern annotations as supervisory data, to train the first ML based model to classify latent vectors of at least one sequence to one or more classes of movement patterns.

39. The method of claim 37, wherein said classes of movement patterns are selected from a list of traversal patterns consisting of short scooting, rapid long scooting, performance of routine turns, performance of C-turns, a pattern of immobility, and a pattern of intermittent mobility.

40. The method of claim 37, wherein said classes of movement patterns are selected from a list of organ movement patterns consisting of: an eye movement pattern, a heart movement pattern, a fin movement pattern, and a limb movement pattern.

41. The method of claim 34, wherein the behavioral indicator is selected from a list consisting levels of: anxiety of the animal, arousal of the animal, responsiveness of the animal to a visual stimulus, responsiveness of the animal to an odor stimulus, responsiveness of the animal to an acoustic stimulus, motoric disability of the animal, appetite of the animal, sleepiness of the animal.

42. The method of claim 34, wherein determining efficacy of the treatment comprises:
comparing a pre-treatment value of the behavioral indicator, with a post-treatment value of the behavioral indicator; and
calculating a statistical significance value, representing correlation between application of the predetermined substance and the behavioral indicator, based on said comparison, to determine efficacy of the treatment.

43. The method of claim 35, wherein extracting the motion features comprises:
identifying one or more body parts of the depicted fish in said images;
applying a second ML based model on the identified body parts, to do determine locations of specific points of the fish, at sub-pixel resolution;
fitting the determined locations in a quadratic curve;
quantifying motion of the at least one body part based on said fitting; and
calculating a value of at least one motion feature based on said quantification.

44. The method of claim 43, wherein quantifying motion of the at least one body part is selected from: (i) computing a rate of tail strokes, based on location of one or more specific points of the fish on the quadratic curve; and (ii) computing an amplitude of tail strokes, based on location of one or more specific points of the fish on the quadratic curve.

45. The method of claim 44, further comprising:
identifying a sub-pixel centroid location of a head of the depicted fish, and
computing the rate of tail strokes, further based on the identified centroid location.

46. A system for determining efficacy of treatment, the system comprising:
at least one camera, configured to obtain images depicting motion of an animal, wherein said animal is treated with a predetermined substance;
a non-transitory memory device wherein modules of instruction code are stored; and
at least one processor associated with the memory device, and configured to execute the modules of instruction code, whereupon execution of said modules of instruction code, the at least one processor is configured to:
receive, from the at least one camera, images depicting motion of the animal;
extract, from said images, a plurality of motion features representing motion of at least one specific body part of the animal;
apply a dimensionality reduction algorithm on the plurality of motion features, to obtain a latent vector representing the plurality of motion features in a latent space, wherein the latent vector comprises a plurality of latent features;
calculate a value of a behavioral indicator, representing a behavior of the animal, based on the latent features of the latent vector; and
determine efficacy of the treatment based on the behavioral indicator value.

47. The system of claim 46, wherein the animal is a fish, and wherein said body parts are selected from a head of the fish, a tail of the fish, an eye of the fish and a heart of the fish.

48. The system of claim 46, wherein the plurality of motion features are selected from at least one of: (a) a list of tail motion features consisting of: a frequency of tail motions, an amplitude of tail motions, an angle of tail motions, a number of tail motions in a predetermined timeframe, a balance of tail motions between a left side and a right side of the fish; (b) a list of head motion features, said list consisting of: a frequency of head motions, an angle of head motions, and a number of head motions within a predefined timeframe; (c) a list consisting of a frequency of fin motions, an amplitude of fin motions, an angle of eye motions, and a frequency of eye motions; and (d) a list of swim interval features, said list consisting of: a duration of a swim episode, and an interval between swim episodes.

49. The system of claim 46, wherein said at least one processor is further configured to:
apply a pretrained, first machine learning (ML) based model, to classify the latent vector to one or more classes of movement patterns; and
calculate the behavioral indicator value based on said classification.

50. The system of claim 49, wherein said at least one processor is further configured to perform training of the first ML based model, said training comprising:
receiving a training set, comprising a plurality of time-based sequences of one or more latent vectors;
obtaining a plurality of movement pattern annotations, corresponding to the plurality of latent vector sequences; and
using the plurality of movement pattern annotations as supervisory data, to train the first ML based model to classify latent vectors of at least one sequence to one or more classes of movement patterns.

51. The system of claim 49, wherein said classes of movement patterns are selected from a list of traversal patterns consisting of short scooting, rapid long scooting, performance of routine turns, performance of C-turns, a pattern of immobility, and a pattern of intermittent mobility.

52. The system of claim 49, wherein said classes of movement patterns are selected from a list of organ movement patterns consisting of: an eye movement pattern, a heart movement pattern, a fin movement pattern, and a limb movement pattern.

53. A method of screening for a compound suitable for treating a psychological state in a subject in need thereof by at least one processor, the method comprising:
administering an animal with an effective amount of the compound;
measuring a plurality of motion features, each representing a specific quantified motion characteristic of at least one specific body part of the animal;
determining a latent vector representing the plurality of motion features in a latent space, wherein the latent vector comprises a plurality of latent features;
calculating a value of a behavioral indicator, representing a behavior of the administered animal, based on the latent features of the latent vector,
wherein a behavioral indicator of the animal administered with the compound being equal to, or greater than a pre-determined threshold, is indicative of the compound being suitable for treating the psychological state in a subject in need thereof.
